SEQUENCE LISTING (43 sequences)

SEQ ID NO: 1 - protein, 4 aa, Artificial Sequence
  Synthetic peptide
AGES

SEQ ID NO: 2 - protein, 4 aa, Artificial Sequence
  Synthetic peptide
SKDP

SEQ ID NO: 3 - protein, 6 aa, Artificial Sequence
  Synthetic peptide
SDKPDM

SEQ ID NO: 4 - protein, 5 aa, Artificial Sequence
  Synthetic peptide
QAGES

SEQ ID NO: 5 - protein, 6 aa, Artificial Sequence
  Synthetic peptide
KQAGES

SEQ ID NO: 6 - protein, 7 aa, Artificial Sequence
  Synthetic peptide
EKQAGES

SEQ ID NO: 7 - protein, 8 aa, Artificial Sequence
  Synthetic peptide
QEKQAGES

SEQ ID NO: 8 - protein, 14 aa, Artificial Sequence
  Synthetic peptide
LKKTETQEKQAGES

SEQ ID NO: 9 - protein, 6 aa, Artificial Sequence
  Synthetic peptide
LKKTET

SEQ ID NO: 10 - protein, 6 aa, Artificial Sequence
  Synthetic peptide
LKKTNT

SEQ ID NO: 11 - protein, 7 aa, Artificial Sequence
  Synthetic peptide
KLKKTET

SEQ ID NO: 12 - protein, 7 aa, Artificial Sequence
  Synthetic peptide
LKKTETQ

SEQ ID NO: 13 - protein, 27 aa, Artificial Sequence
  Synthetic peptide
LKKTETQEKNPLPSKETIEQEKQAGES

SEQ ID NO: 14 - protein, 43 aa, Artificial Sequence
  Synthetic peptide
SDKPDMAEIEKFDKSKLKKTETQEKNPLPSKETIEQEKQAGES

SEQ ID NO: 15 - protein, 4 aa, Artificial Sequence
  Synthetic peptide
SDKP

SEQ ID NO: 16 - protein, 6 aa, Artificial Sequence
  Synthetic peptide
SDKPDM

SEQ ID NO: 17 - protein, 16 aa, Artificial Sequence
  Synthetic peptide
SDKPDMAEIEKFDKSK

SEQ ID NO: 18 - protein, 12 aa, Artificial Sequence
  Synthetic peptide
DMAEIEKFDKSK

SEQ ID NO: 19 - protein, 6 aa, Artificial Sequence
  Synthetic peptide
LKKTET

SEQ ID NO: 20 - protein, 10 aa, Artificial Sequence
  Synthetic peptide
SDKPLKKTET

SEQ ID NO: 21 - protein, 22 aa, Artificial Sequence
  Synthetic peptide
SDKPDMAEIEKFDKSKLKKTET

SEQ ID NO: 22 - protein, 39 aa, Artificial Sequence
  Synthetic peptide
SKDPDMAEIEKFDKSKLKKTETQEKNPLPSKETIEQEKQ

SEQ ID NO: 23 - protein, 34 aa, Artificial Sequence
  Synthetic peptide
DMAEIKFDKSKLKKTETQEKNPLPSKETIEQEKQ

SEQ ID NO: 24 - protein, 39 aa, Artificial Sequence
  Synthetic peptide
DMAEIEKFDKSKLKKTETQEKNPLPSKETIEQEKQAGES

SEQ ID NO: 25 - protein, 27 aa, Artificial Sequence
  Synthetic peptide
LKKTETQEKNPLPSKETIEQEKQAGES

SEQ ID NO: 26 - protein, 21 aa, Artificial Sequence
  Synthetic peptide
QEKNPLPSKETIEQEKQAGES

SEQ ID NO: 27 - protein, 17 aa, Artificial Sequence
  Synthetic peptide
QEKNPLPSKETIEQEKQ

SEQ ID NO: 28 - protein, 4 aa, Artificial Sequence
  Synthetic peptide
AGES

SEQ ID NO: 29 - protein, 43 aa, Artificial Sequence; identical to SEQ ID NO: 14
  Synthetic peptide

SEQ ID NO: 30 - protein, 4 aa, Artificial Sequence
  Synthetic peptide
SDKP

SEQ ID NO: 31 - protein, 6 aa, Artificial Sequence
  Synthetic peptide
SDKPDM

SEQ ID NO: 32 - protein, 16 aa, Artificial Sequence
  Synthetic peptide
SDKPDMAEIEKFDKSK

SEQ ID NO: 33 - protein, 12 aa, Artificial Sequence
  Synthetic peptide
DMAEIEKFDKSK

SEQ ID NO: 34 - protein, 6 aa, Artificial Sequence
  Synthetic peptide
LKKTET

SEQ ID NO: 35 - protein, 12 aa, Artificial Sequence
  Synthetic peptide
SDKPDMLKKTET

SEQ ID NO: 36 - protein, 23 aa, Artificial Sequence
  Synthetic peptide
SDKPDMAEIEKFDSKSKLKKTET

SEQ ID NO: 37 - protein, 39 aa, Artificial Sequence
  Synthetic peptide
SDKPDMAEIEKFDKSKLKKTETQEKNPLPSKETIEQEKQ

SEQ ID NO: 38 - protein, 35 aa, Artificial Sequence
  Synthetic peptide
DMAEIEKFDKSKLKKTETQEKNPLPSKETIEQEKQ

SEQ ID NO: 39 - protein, 39 aa, Artificial Sequence; identical to SEQ ID NO: 24
  Synthetic peptide

SEQ ID NO: 40 - protein, 27 aa, Artificial Sequence
  Synthetic peptide
LKKTETQEKNPLPSKETIEQEKQAGES

SEQ ID NO: 41 - protein, 27 aa, Artificial Sequence
  Synthetic peptide
LKKTETQEKNPLPSKETIEQEKQAGES

SEQ ID NO: 42 - protein, 20 aa, Artificial Sequence
  Synthetic peptide
EKNPLPSKETIEQEKQAGES

SEQ ID NO: 43 - protein, 16 aa, Artificial Sequence
  Synthetic peptide
EKNPLPSKETIEQEKQ

(19) United States
(12) Patent Application Publication
(10) Pub. No.: US 2011/0251128 A1
(43) Pub. Date: Oct. 13, 2011

(54) THYMOSIN Beta4 PEPTIDES PROMOTE TISSUE REGENERATION

(75) Inventors: ILDIKO BOCK-MARQUETTE, Dallas, TX (US); J. MICHAEL DIMAIO, Dallas, TX (US)

(73) Assignee: The Board of Regents of the University of Texas System

(21) Appl. No.: 13/035,588

(22) Filed: Feb. 25, 2011

Related U.S. Application Data
(60) Provisional application No. 61/308,618, filed on Feb. 26, 2010.

(51) Int. Cl. A61K 38/07; C07K 7/06; C07K 7/08; A61K 38/08; A61P 9/10; C12N 5/071; C12N 5/0797; C12N 5/077; A61P 29/00; A61P 9/00; C07K 5/103; A61K 38/10

(52) U.S. Cl. 514/16.4; 530/330; 530/328; 530/329; 530/327; 435/375; 514/21.9; 514/21.7

(57) ABSTRACT

The present invention relates to thymosin β-4 peptides and analogs thereof that can promote tissues regeneration, particularly cardiac tissue.

This application claims benefit of priority under 35 U.S.C. §119(e) to U.S. Provisional Application Ser. No. 61/308,618, filed Feb. 26, 2010, the entire contents of which are hereby incorporated by reference.

This invention was made with government support under grant no. 5 K08 HD054872-02 awarded by the National Institutes of Health. The government has certain rights in the invention.

### BACKGROUND OF THE INVENTION

I. Field of the Invention

The present invention relates generally to the fields of cell biology, developmental biology and cardiology. More particularly, it concerns methods and compositions relating to the treatment or prevention of damage to cardiac tissue.

II. Description of Related Art

Coronary artery disease results in acute occlusion of cardiac vessels leading to loss of dependent myocardium. Such events are one of the leading causes of death in the Western world. Because the heart is incapable of sufficient muscle regeneration, survivors of myocardial infarctions typically develop chronic heart failure with over ten million cases in the United States alone. While more commonly affecting adults, heart disease in children is the leading non-infectious cause of death in the first year of life and often involves abnormalities in cardiac cell specification, migration or survival.

There are many causes of myocardial and coronary vessel and tissue injuries, including but not limited to myocardial ischemia, clotting, vessel occlusion, infection, developmental defects or abnormalities and other such myocardial events. Myocardial infarction results from blood vessel disease in the heart. It occurs when the blood supply to part of the heart is reduced or stopped (caused by blockage of a coronary artery, as one example). The reduced blood supply causes injuries to the heart muscle cells and may even kill heart muscle cells. The reduction in blood supply to the heart is often caused by narrowing of the epicardial blood vessels due to plaque. These plaques may rupture causing hemorrhage, thrombus formation, fibrin and platelet accumulation and constriction of the blood vessels.

In addition, there are a number of drugs, devices and medical procedures which are utilized to unclog or increase blood flow through arteries and other blood vessels. However, unclogging of blood vessels sometimes permits a large amount of blood, containing oxygen, free radicals and other chemicals, to rush into a tissue site with a potential for causing damage to the tissue.

Recent evidence suggests that a population of extracardiac or intracardiac stem cells may contribute to maintenance of the cardiomyocyte population under normal circumstances. Efforts to promote cardiac repair by introduction or recruitment of exogenous stem cells hold promise but typically involve isolation and introduction of autologous or donor progenitor cells. While the stem cell population may maintain a delicate balance between cell death and cell renewal, it is insufficient for myocardial repair after acute coronary occlusion. Introduction of isolated stem cells may improve myocardial function, but this approach has been controversial, and requires isolation of autologous stem cells or use of donor stem cells along with immunosuppression. Efforts to coax pluripotent embryonic stem cells into a cardiomyocyte lineage remain unsuccessful. Technical hurdles of stem cell delivery and differentiation have thus far prevented broad clinical application of cardiac regenerative therapies.

There remains a need in the art for methods and compositions for treating or preventing cardiac tissue damage.

### SUMMARY OF THE INVENTION

Thus, in accordance with the present invention, there is provided a peptide of less than 15 residues in length and comprising the sequence AGES (SEQ ID NO:1), SKDP (SEQ ID NO:2) or both. The peptide may be comprise SKDP, SDKPDM (SEQ ID NO:3), AGES, QAGES (SEQ ID NO:4), KQAGES (SEQ ID NO:5), EKQAGES (SEQ ID NO:6), or QEKQAGES (SEQ ID NO:7). The peptide may comprise less than 13, less than 12, less than 11, less than 10, less than 9, less than 8, less than 7, or less than 6 residues. The peptide may consists of SKDP, SDKPDM, AGES, QAGES, KQAGES, EKQAGES, QEKQAGES or LKKTETQEKQAGES (SEQ ID NO:8). The peptide may partially or wholly comprise D amino acid residues. The peptide may comprise a SDKPDM motif, and optionally include a modification selected from the group deacetylization, methionine oxidation.

In another embodiment, there is provided a method of promoting cardiac repair comprising administering to a subject a peptide of less than 15 residues in length and comprising the sequence AGES, SKDP or both. The peptide may be comprise SKDP, AGES, SDKPDM, QAGES, KQAGES, EKQAGES, or QEKQAGES. The peptide may comprise less than 13, less than 12, less than 11, less than 10, less than 9, less than 8, less than 7, or less than 6 residues. The peptide may consist of SKDP, SDKPDM, AGES, QAGES, KQAGES, EKQAGES, QEKQAGES or LKKTETQEKQAGES. The peptide may partially or wholly comprise D amino acid residues. The peptide may comprise a SDKPDM motif, and optionally include a modification selected from the group deacetylization, methionine oxidation.

The subject may a human subject, such as one that has suffered a myocardial infarct. Administering may comprise intravenous, intraarterial, intracardiac, ocular, or topical administration. Administering may comprise multiple administrations, such as those provided over 3, 4, 5, 6, 7, 14, 21 or 28 days, or those provided over 3, 4, 5, 6, 7, 14, 21 or 28 days post-infarct. Administration may comprise continuous infusion over a period of 1-24 hours, or continuous infusion over a period of 1-24 hours post-infarct. The method may further comprise assessing cardiac function in said subject. The method may further comprise providing to said subject a second cardiac therapeutic agent, for example, where the subject has suffered a myocardial infarct, the second therapeutic agent may be oxygen, aspirin, glyceryl nitrate, a thrombolytic agent, a β blocker, an anticoagulant, or an antiplatelet agent.

In yet another embodiment, there is provided a method of activating a progenitor cell comprising contacting said cell with a peptide of less than 15 residues in length and comprising the sequence AGES, SKDP or both. The progenitor cell may be a heart progenitor cell, a brain progenitor cell, a lung progenitor cell, a skeletal muscle progenitor cell, a kidney progenitor cell, a liver progenitor cell, a pancreatic progenitor cell, a spleen progenitor cell, a skin progenitor cell, or a bone marrow progenitor cell.

In still another embodiment, there is provided a method of reducing inflammation in a subject comprising administering to said subject a peptide of less than 15 residues in length and comprising the sequence AGES, SKDP or both. The peptide may be comprise AGES, SKDP, SDKPDM, QAGES, KQAGES, EKQAGES, or QEKQAGES. The peptide may comprise less than 13, less than 12, less than 11, less than 10, less than 9, less than 8, less than 7, or less than 6 residues. The peptide may consist of SKDP, SDKPDM, AGES, QAGES, KQAGES, EKQAGES, QEKQAGES or LKKTETQEKQAGES. The peptide may partially or wholly comprise D amino acid residues.

In still yet another embodiment, there is provided a method of reducing the size of a myocardial infarct comprising administering to a subject that has suffered a myocardial infarct a peptide of less than 15 residues in length and comprising the sequence AGES. The peptide may be comprise AGES, SKDP, SDKPDM, QAGES, KQAGES, EKQAGES, or QEKQAGES. The peptide may comprise less than 13, less than 12, less than 11, less than 10, less than 9, less than 8, less than 7, or less than 6 residues. The peptide may consists of SKDP, SDKPDM, AGES, QAGES, KQAGES, EKQAGES, QEKQAGES or LKKTETQEKQAGES. The peptide may partially or wholly comprise D amino acid residues.

A further embodiment comprising a method of delivering an agent to a cell comprising providing to said cell a peptide of less than 15 residues in length and comprising the sequence AGES, SKDP, or both, said peptide conjugated to said agent. The agent may be an siRNA, an miRNA, an antisense molecule, a organopharmaceutical, or another peptide. The peptide may be comprise AGES, SKDP, SDKPDM QAGES, KQAGES, EKQAGES, or QEKQAGES. The peptide may comprise less than 13, less than 12, less than 11, less than 10, less than 9, less than 8, less than 7, or less than 6 residues. The peptide may consist of SKDP, SDKPDM, AGES, QAGES, KQAGES, EKQAGES, QEKQAGES or LKKTETQEKQAGES. The peptide may partially or wholly comprise D amino acid residues.

It is contemplated that any method, compound or composition described herein can be implemented with respect to any other method or composition described herein.

For any embodiment that refers to a composition, a compound may be used, and vice versa, unless specifically noted otherwise.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. In vitro comparison of peptide constructs to full-length TB4 on embryonic cardiac cells using in vitro collagen gel migration assay. (FIG. 1A) Amino acid sequence of full length TB4 and synthesized peptides. (FIG. 1B) Migration distance (cardiac endothelial cells) and beating frequency (myocardial cells) analyses at days three and nine indicate, the C-terminal variable region of TB4 (#17) increases endothelial cell migration and myocardial beating frequency in higher, while construct #14 in similar extent than full-length TB4. Means and 95% confidence limits are shown. #, P<0.005 (n=3/each). (FIG. 1C) Summary of the in vitro effects of various TB4 domains on cardiac endothelial cell migration and myocyte beating.

FIGS. 2A-N. Peptide #14 and #17 treatment improves myocardial function and alters scar formation and myocyte size in vivo after coronary ligation in adult mice. (FIG. 2A) Representative echocardiographic M-mode images of left ventricles after coronary ligation with peptide #14 (#14), peptide #17 (#17), TB4 or PBS treatment. (FIGS. 2B-C) Distribution of left ventricular fractional shortening (FS) (FIG. 2B) or ejection fraction (EF) (FIG. 2C) at 3 weeks after coronary ligation with #14 (n=17), #17, (n=16), TB4 (n=14) or PBS (n=11) treatment. Bars indicate means. (FIG. 2D) Echocardiographic measurements for intraperitoneal and intracardiac administration of #14, #17, TB4 or PBS at 3 weeks. Means and 95% confidence limits are shown. Asterisk, P<0.01. (FIGS. 2E-L), Representative trichrome stain of transverse heart sections at comparable levels 21 days after coronary ligation and #14 (FIG. 2E, (FIG. 2I), #17 (FIG. 2F, FIG. 2J), TB4 (FIG. 2G, FIG. 2K) or PBS (FIG. 2H, FIG. 2L) treatment delivered intracardiac and intraperitoneally. FIGS. 2I-L are higher magnifications of FIGS. 2E-H boxed areas respectively. Collagen in scar is indicated in blue and myocytes in red. LV, left ventricle; bars: 300 mm. (FIG. 2M) Estimated scar volume of hearts after coronary ligation and #14, #17, TB4 and PBS treatment. (FIG. 2N) Peptide #14, #17 and TB4 significantly increase the size of surviving cardiomyocytes at the zone of infarction. Bars indicate standard deviation at 95% confidence limits.

FIGS. 3A-E. Effect of Peptide #14 and #17 on cardioprotection in adult pigs after cardiac infarction. (FIGS. 3A-E) Quantification revealed significant increase in global (FIG. 3B) and regional (FIG. 3C) myocardial function and decrease in infarct size (FIG. 3A yellow area, FIG. 3D gray bars) after #17 retroinfusion when compared to PBS treated controls in adult infarcted pigs. AAR/left ventricle (LV) did not differ significantly between groups (FIG. 3A blue area, FIG. 3D black bars). (FIG. 3E) Myeloperoxidase (MPO) activity in non-ischemic, AAR (non-infarcted), and infarcted regions after retroinfusion of peptide #14 and #17 indicate reduction of post-ischemic inflammation. Bars indicate standard deviation at 95% confidence limits Asterisk, P<0.05 (n=5/each).

FIGS. 4A-B. Biodistribution of AGES in adult human cardiac cells and mice. (FIG. 4A) In vivo PET-scan images and diagrams indicate high uptake of AGES into the heart when compared to brain, lungs, liver and skeletal muscle and to “no peptide” control (n=3/each treatment). (FIG. 4B) Nanogold labeled AGES conjugate enters the nucleus of adult human cardiomyocytes as early as ten minutes after extracellular administration. In contrary to cardiomyocytes, adult human coronary endothelial cells internalize peptide #17 only twenty minutes after administration into their cytoplasm but not into the nucleus. Immunhistostaing with sarcomeric α-actinin or pCytokeratin (green) indicate origin of cells; bar: 100 mm.

FIGS. 5A-L. Peptide #14 and #17 promote cell survival and initiate cardiac vessel formation in vivo after coronary artery ligation in adult mice. (FIGS. 5A-D) TUNEL-positive cells (bright green) double-labeled with anti-sarcomeric α-actinin antibody (red) to mark positive cardiomyocytes 24 h after coronary ligation and #14, #17, TB4 or PBS treatment; bars: 200 mm. (FIG. 5E) Quantification of TUNEL positive cardiomyocytes indicates significant decrease of myocardial cell death at the area of risk after #14, #17 and TB4 treatment. (FIGS. 5F-I) Immunohistochemistry using smooth muscle α-actin (bright green) specific antibodies revealed increase of mature vessel structures at the margin of the scar 21 days after #14 and #17 treatment (FIG. 5F, FIG. 5G) similar to full-length TB4 (FIG. 5H), when compared to PBS (FIG. 5I). m, intact myocardium; s, infarcted scar; v, mature vessels; bars: 400 mm. (FIG. 5J) Number of vessels increases significantly after #14 and #17 treatment compared to PBS control. Bars indicate standard deviation at 95% confidence limits (n=3/each). (FIG. 5K) Western blot using anti-phosphoS473-Akt on heart lysates after coronary ligation indicate increased Akt activation 24 hours after #14, #17 and TB4 treatment, while Akt levels reamined unaltered. Bars indicate standard deviation at 95% confidence limits (n=3/each). (FIG. 5L) Densitometric analyses of Western blot results of adult cardiac tissue indicate increase in VEGF expression and Protein Kinase C activation twenty-one days after #14 and #17 injection when compared to PBS treatment in vivo. Density units were normalized to GAPDH loading control. Bars indicate standard deviation at 95% confidence limits (n=3/each).

FIGS. 6A-J. Alterations of Connexin43 distribution and Wt-1 positive cardiac progenitors after #14 and #17 treatment in infarcted adult mice. (FIGS. 6A-E) Immunohistochemistry at the infarction border (FIGS. 6A-D (green)) and quantification of Wt-1 positive progenitor cells (FIG. 6E) show significant increase 21 days after peptide #17 and TB4 treatment. Bars indicate standard deviation at 95% confidence limits (n=3/each). (FIGS. 6F-J) Connexin43 expression and localization at the border zone of cardiac infarction. Immunohistological analysis of Connexin43 indicates, in #17 and TB4 treated hearts Cx43 was mainly localized to the intercalated discs (FIGS. 6G-H white arrowheads), whereas in #14 and PBS treated hearts Cx43 was mostly distributed around the myocytes (FIG. 6F, FIG. 6I white arrows). (FIG. 6J) Western blot analysis of Cx43 expression and activation using Cx43 and pS368-Cx43 antibodies show significant increase in Cx43 level and decrease in Cx43 phosphorylation by #17 and TB4 treatment 21 days after infarction. Density units were normalized to Coomassie staining and GAPDH loading control. Bars indicate standard deviation at 95% confidence limits (n=3/each). sc, scar; m, myocardium; bars: 100 mm.

FIG. 7. Pathology of post-ischemic cardiac remodeling in humans.

FIG. 8. In vitro comparison of peptide constructs to full-length TB4 on embryonic cardiac cells using collagen gel migration assay. Outflow tract from embryonic day 11.5 wild-type mice was placed endothelial surface facing down onto hydrated rat tail type I collagen matrices. Migration distance and beating frequency analysis of cardiac endothelial and myocardial cells at days three and nine indicate the C-terminal variable region of TB4 (#17 AGES (red)) increases cell migration and beating frequency respectively when compared to full-length TB4. Additionally, our results suggest acetylation of the N terminal domain (#1, #2 compared to #4 and #5), or addition of 6Met (#2) inhibit myocyte beating in vitro. Actin binding domain LKKTET (#8) alone increases beating frequency. This was suppressed by the N-terminal variable (#9) and helix domains (#11 and #12) and by the addition of the C-terminal helix (#11) suggesting N- and C-terminal helixes negatively affect cardiac cell behavior.

FIGS. 9A-B. In vivo biodistribution of peptide #17 and “no peptide” conjugate (control) in adult mice. A-B, Percent of systemically (intra venous (i.v.)) injected #17 (FIG. 9A) and “no peptide” (FIG. 9B) conjugate per gram (n=3/each).

FIGS. 10A-K. (FIG. 10A) Peptide #3 does not alter cardiac function in vivo after cardiac infarction in adult mice. Bars indicate standard deviation at 95% confidence limits. (FIGS. 10B-K) Similar to PBS peptide, #3 fails to alter the number of mature coronary vessels after cardiac infarction in mice when compared to full length TB4. (FIGS. 10C-K) Immunohistochemistry with smooth muscle α-actin (FIG. 10C, FIG. 10F, FIG. 10I green) and PECAM-1 (FIG. 10D, FIG. 10G, FIG. 10J red) antibodies at the infarction border zone two weeks after #3 treatment. FIG. 10E, FIG. 10H and FIG. 10K are DAPI stain of FIG. 10C, FIG. 10D, FIG. 10F, FIG. 10G, FIG. 10I and FIG. 10J. (FIG. 10B) Number of mature vessels increases after TB4 but remains unaltered after #3 treatment compared to PBS control. Bars indicate standard deviation at 95% confidence limits (n=3/each); bars: 100 mm.

FIGS. 11A-H. (FIGS. 11A-G) Peptide #3 fails to increase Wt-1 positive progenitor cells at the infarction border. (FIG. 11A) Quantification of Wt-1 positive cells. (FIGS. 11B-G) Immunohistochemical analysis using Wt-1 antibody (green) at the infarction border two weeks after #3, TB4 and PBS injections (white arrows point at Wt-1 positive cells). FIG. 11C, FIG. 11E and FIG. 11G are DAPI staining of FIG. 11B, FIG. 11D and FIG. 11F. ic, infarction core; mc, myocardium; bars: 100 mm. (FIG. 11H) Western blot indicates N-Cadherin is slightly upregulated in the infarcted core by #17 and TB4 treatment and unchanged following #14 injection 21 days after cardiac infarction. Bars indicate standard deviation at 95% confidence limits (n=3/each).

### DESCRIPTION OF ILLUSTRATIVE EMBODIMENTS

In accordance with the present invention, a method of treating or preventing cardiac tissue damage by administering an effective amount of a composition comprising a tissue damage-reducing or preventing peptide comprising at least one of thymosin β4 peptide as defined herein. The composition is administered to said tissue during at least one of before, during or after tissue damage occurs.

The present invention is based on a discovery that peptides such as thymosin β4 (Tβ4) and other, e.g., actin-sequestering peptides or peptide fragments which may contain amino acid sequence LKKTET (SEQ ID NO:9) or LKKTNT (SEQ ID NO:10) or conservative variants thereof (hereinafter sometimes referred to as a “tissue damage-preventing or -reducing peptide(s)”), promote healing or prevention of cardiac or other tissue damage and other changes associated with an increase in blood flow. Such tissue damage-preventing peptides comprise at least one of Thymosin β4 (Tβ4), an isoform of Tβ4, an N-terminal fragment of Tβ4, a C-terminal fragment of Tβ4, Tβ4 sulfoxide, an LKKTET peptide, an LKKTNT peptide, an actin-sequestering peptide, an actin binding peptide, an actin-mobilizing peptide, an actin polymerization-modulating peptide, or a conservative variant thereof. Included are N- or C-terminal fragments or variants, which may or may not include KLKKTET (SEQ ID NO:11) and LKKTETQ (SEQ ID NO:12). Tβ4 has been suggested as being a factor in angiogenesis in rodent models. However, there heretofore has been no known indication that such properties may be useful in treating tissue damage caused by an increase in blood flow. Without being bound to any particular theory, these peptides may have the capacity to promote repair, healing and prevention by having the ability to induce terminal deoxynucleotidyl transferase (a non-template directed DNA polymerase), to decrease and modulate the levels of one or more inflammatory cytokines or chemokines, and to act as a chemotactic and/or angiogenic factor for cells and thus heal and prevent tissue damage caused by an increase in blood flow.

The invention is particularly useful in conjunction with use of agents (e.g., drugs, devices or procedures) utilized to unclog or increase blood flow through arteries and other blood vessels. In order to prevent or treat tissue damage occurring subsequent to affecting an increase in blood flow through a blood vessel which is in communication with the tissue, the tissue damage-preventing or -reducing peptide can be administered before, during and/or after affecting the increase in blood flow.

Agents which may be utilized to affect an increase in blood flow through a blood vessel include, but are not limited to, aspirin, tPA, streptokinase, plasminogen, anti-clotting agents, antistreplase, reteplase, tenecteplase and/or heparin. The tissue damage-preventing or -reducing peptide can be administered before, during and/or after blood flow is increased in conjunction therewith. Amounts of such agents which are effective in increasing blood flow through blood vessels are included within the range of 0.001-1,000 mg. The invention also is applicable to compositions comprising such blood flow-increasing agents and a tissue damage-preventing or -reducing peptide.

Devices and procedures which may be utilized to affect an increase in blood flow through a blood vessel include, but are not limited to, arterial stents, venous stents, cardiac catheterizations, carotid stents, aortic stents, pulmonary stents, angioplasty, bypass surgery and/or neurosurgery. The tissue damage-preventing or -reducing peptide can be administered before, during and/or after blood flow is increased in conjunction therewith.

Indications to which the invention may be applicable include, but are not limited to, trauma induced ischemia (e.g., cardio), disease induced ischemia, inflammation, idiopathic ischemia and/or stroke. The tissue damage-preventing or -reducing peptide can be administered before, during and/or after blood flow is increased in conjunction therewith.

Tissue damage-preventing or -reducing peptides as described herein, can prevent and/or limit the apoptic death of brain and other neurovascular cells and tissues following ischemic, infectious, pathological, toxic or traumatic damage by upregulating metabolic and signaling enzymes such as the phosphatidylinositol 3-kinase (P13-K)/Akt (protein kinase β) pathway and protein kinase C. Upregulating P13-K/Akt and downstream phosphorylated Bad and proline rich Akt survival kinase protects neuronal cells during hypoxic insults. In addition, tissue damage-preventing or -reducing peptides as described herein, by virtue of their ability to downregulate inflammatory cytokines such as IL-18 and chemokines such as IL-8 and enzymes such as caspace 2, 3, 8 and 9 protects neuronal cells and facilitates healing of nervous tissue.

As noted above, the tissue damage-preventing or -reducing peptide may be administered before, during and/or after affecting an increase in blood flow through a blood vessel which is in communication with the tissue. Delivery pathways include, but are not limited to, parenteral, oral, nasal, pulmonary, intracardiac, intravenous, transdermal and/or liposomal.

Thymosin β4 was initially identified as a protein that is up-regulated during endothelial cell migration and differentiation in vitro. Thymosin β4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

In accordance with one embodiment, the invention is a method of treatment for promoting healing and prevention of damage and inflammation associated with tissue damage caused by an increase in blood flow comprising administering to a subject in need of such treatment an effective amount of a composition comprising a tissue damage-reducing peptide comprising amino acid sequence LKKTET or LKKTNT, or a conservative variant thereof having a tissue damage-reducing activity, preferably Thymosin β4, an isoform of Thymosin β4, or an antagonist of Thymosin β4. The invention may also utilize oxidized Tβ4.

Compositions which may be used in accordance with the present invention include Thymosin β4 (Tβ4), Tβ4 isoforms, oxidized Tβ4, polypeptides or any other actin sequestering or bundling proteins having actin binding domains, or peptide fragments which may or may not comprise or consist essentially of the amino acid sequence LKKTET or LKKTNT or conservative variants thereof, having tissue damage-reducing activity. International Application Serial No. PCT/US99/17282, incorporated herein by reference, discloses isoforms of Tβ4 which may be useful in accordance with the present invention as well as amino acid sequence LKKTET and conservative variants thereof, which may be utilized with the present invention. International Application Serial No. PCT/GB99/00833 (WO 99/49883), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET, LKKTNT, LKKTETQ:, or KLKKTET peptides and fragments comprising or consisting essentially of LKKTET, or LKKTNT or LKKTETQ or KLKKTET, conservative variants thereof having tissue damage-reducing activity, as well as oxidized Thymosin β4 and other tissue damage-preventing or -reducing peptides as described herein.

In one embodiment, the invention provides a method for healing and preventing inflammation and damage in a subject by contacting the tissue site with an effective amount of a tissue damage-reducing composition which contains Tβ4 or a Tβ4 isoform or other tissue damage-preventing or -reducing peptides as described herein. The contacting may be direct or systemically. Examples of contacting the damaged site include contacting the site with a composition comprising the tissue damage-preventing or -reducing peptide alone, or in combination with at least one agent that enhances penetration, or delays or slows release of tissue damage-preventing or -reducing peptides into the area to be treated. The administration may be directly or systemically. Examples of administration include, for example, direct application, injection or infusion, with a solution, lotion, salve, gel cream, paste spray, suspension, dispersion, hydrogel, ointment, foam, oil or solid comprising a tissue damage-preventing or -reducing peptide as described herein. Administration may include, for example, intravenous, intraperitoneal, intramuscular or subcutaneous injections, or inhalation, transdermal or oral administration of a composition containing the tissue damage-preventing or -reducing peptide, etc. A subject may be a mammal, preferably human.

The tissue damage-preventing or -reducing peptide may be administered in any suitable tissue damage-reducing or -preventing amount. For example, tissue damage-preventing or -reducing peptide may be administered in dosages within the range of about 0.0001-1,000,000 micrograms, more preferably about 0.01-5,000 micrograms, still more preferably about 0.1-50 micrograms, most preferably in amounts within the range of about 1-30 micrograms.

A composition in accordance with the present invention can be administered daily, every other day, etc., with a single administration or multiple administrations per day of administration, such as applications 2, 3, 4 or more times per day of administration.

Tβ4 isoforms have been identified and have about 70%, or about 75%, or about 80% or more homology to the known amino acid sequence of Tβ4. Such isoforms include, for example, Tβ4ala, Tβ9, Tβ10, Tβ11, Tβ12, Tβ13, Tβ14 and Tβ15. Similar to Tβ4, the Tβ10 and Tβ15 isoforms have been shown to sequester actin. T.beta.4, Tβ10 and Tβ15, as well as these other isoforms share an amino acid sequence, LKKTET or LKKTNT, that appears to be involved in mediating actin sequestration or binding. Although not wishing to be bound to any particular theory, the activity of Tβ4 isoforms may be due, in part, to the ability to regulate the polymerization of actin. .beta.-thymosins appear to depolymerize F-actin by sequestering free G-actin. Tβ4's ability to modulate actin polymerization may therefore be due to all, or in part, its ability to bind to or sequester actin via the LKKTET or LKKTNT sequence. Thus, as with Tβ4, other tissue damage-preventing or -reducing proteins which may bind or sequester actin, or modulate actin polymerization, including Tβ4 isoforms having the amino acid sequence LKKTET or LKKTNT, are likely to be effective, alone or in a combination with Tβ4, as set forth herein.

Thus, it is specifically contemplated that known Tβ4 isoforms, such as Tβ4ala, Tβ9, Tβ10, Tβ11, Tβ12, Tβ13, Tβ14 and Tβ15, as well as Tβ4 isoforms not yet identified, will be useful in the methods of the invention. As such Tβ4 isoforms are useful in the methods of the invention, including the methods practiced in a subject. The invention therefore further provides pharmaceutical compositions comprising Tβ4, as well as Tβ4 isoforms Tβ4ala, Tβ9, Tβ10, Tβ11, Tβ12, Tβ13, Tβ14 and Tβ15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET or LKKTNT, for example, can similarly be employed in the methods of the invention. Such proteins include gelsolin, vitamin D binding protein (DBP), profilin, cofilin, adsevertin, propomyosin, fincilin, depactin, Dnasel, vilin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, DNAseI, vilin, fragmin, severin, capping protein, β-actinin and acumentin as set forth herein. Thus, the invention includes the use of a tissue damage-reducing polypeptide which may comprise the amino acid sequence LKKTET or LKKINT (which may be within its primary amino acid sequence) and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

Tβ4 has been localized to a number of tissue and cell types and thus, agents which stimulate the production of Tβ4 or another tissue damage-preventing or -reducing peptide can be added to or comprise a composition to effect tissue damage-preventing or -reducing peptide production from a tissue and/or a cell. Such agents include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF). More preferably, the agent is transforming growth factor β (TGF-β) or other members of the TGF-β superfamily. Compositions of the invention may reduce tissue damage caused by an increase in blood flow by effectuating growth of the connective tissue through extracellular matrix deposition, cellular migration and vascularization.

In accordance with one embodiment, subjects are treated with an agent that stimulates production in the subject of a tissue damage-preventing or -reducing peptide as defined herein.

Additionally, agents that assist or stimulate healing of tissue damage caused by an increase in blood flow event may be added to a composition along with tissue damage-preventing or -reducing peptide. Such agents include angiogenic agents, growth factors, agents that direct differentiation of cells. For example, and not by way of limitation, tissue damage-preventing or -reducing peptides alone or in combination can be added in combination with any one or more of the following agents: VEGF, KGF, FGF, PDGF, TGFβ, IGF-1, IGF-2, IL-1, prothymosin a and thymosin al in an effective amount.

The invention also includes a pharmaceutical composition comprising a therapeutically effective amount of tissue damage-preventing or -reducing peptide in a pharmaceutically acceptable carrier. Such carriers include, inter alia, those listed herein.

The actual dosage, formulation or composition that heals or prevents inflammation, damage and degeneration associated with tissue damage caused by an increase in blood flow may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable formulations include tissue damage-preventing or -reducing peptide at a concentration within the range of about 0.001-50% by weight, more preferably within the range of about 0.01-0.1% by weight, most preferably about 0.05% by weight.

The therapeutic approaches described herein involve various routes of administration or delivery of reagents or compositions comprising the tissue damage-preventing or -reducing compounds of the invention, including any conventional administration techniques to a subject. The methods and compositions using or containing tissue damage-preventing or -reducing compounds of the invention may be formulated into pharmaceutical compositions by admixture with pharmaceutically acceptable non-toxic excipients or carriers.

The invention includes use of antibodies which interact with tissue damage-preventing or -reducing peptides or functional fragments thereof Antibodies which consists essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of an agent which modulates tissue damage-preventing or -reducing peptide gene expression. The term “modulate” refers to inhibition or suppression of tissue damage-preventing or -reducing peptide expression when tissue damage-preventing or -reducing peptide is over expressed, and induction of expression when tissue damage-preventing or -reducing peptide is under expressed. The term “effective amount” means that amount of modulating agent which is effective in modulating tissue damage-preventing or -reducing peptide gene expression resulting in effective treatment. An agent which modulates Tβ4 or tissue damage-preventing or -reducing peptide gene expression may be a polynucleotide for example. The polynucleotide may be an antisense, a triplex agent, or a ribozyme. For example, an antisense directed to the structural gene region or to the promoter region of Tβ4 may be utilized.

In another embodiment, the invention provides a method for utilizing compounds that modulate Tβ4 or tissue damage-preventing or -reducing peptide activity. Compounds that affect Tβ4 or tissue damage-preventing or -reducing peptide activity (e.g., antagonists and agonists) include peptides, peptidomimetics, polypeptides, chemical compounds, minerals such as zincs, and biological agents.

While not be bound to any particular theory, the present invention may promote healing or prevention of inflammation or damage associated with tissue damage caused by an increase in blood flow by inducing terminal deoxynucleotidyl transferase (a non-template directed DNA polymerase), to decrease the levels of one or more inflammatory cytokines, or chemokines, and to act as a chemotactic factor for endothelial cells, and thereby promoting healing or preventing tissue damage caused by an increase in blood flow or other degenerative or environmental factors.

These, and other aspects of the invention, are set out in detail below.

### I. THYMOSIN β4

Thymosin is an actin-binding protein in cells. It promotes the development of immune-system cells. The predominant form of thymosin, thymosin β4, is a member of a highly conserved family of actin monomer-sequestering proteins. β-thymosins are the primary regulators of unpolymerized actin, and are essential for maintaining the small cytoplasmic pool of free G-actin monomers required for rapid filament elongation and allowing for the flux of monomers between the thymosin-bound pool and F-actin.

Thymosin β4 sequesters actin, holding it in a form that is unable to polymerize. Due to its profusion in the cytosol and its ability to bind ATP G-actin but not F-actin, thymosin β4 is regarded as the principal actin-sequestering protein. Tβ4 binds ATP G-monomeric actin in a 1:1 complex where G-actin cannot polymerize. Thymosin β4 (Tβ4) functions like a buffer for monomeric actin as represented in the following reaction:

F-actin⇄G-actin+Tβ4⇄G-actin/Tβ4

Increase in cytosolic concentrations of thymosin β4 increases the concentration of sequestered actin subunits and correspondingly decreases F-actin due to actin filaments being in equilibrium with actin monomers. Furthermore, the inhibitory thymosin which sequesters the actin competes for monomers with profilin.

Actin monomers preferentially bind to either ATP or ADP and both forms of G-actin are capable of polymerization. The affinities of ATP-G-actin and ADP-G-actin monomers for thymosin and profilin vary significantly. Both profilin and thymosin β4 bind ATP monomers with higher affinity than ADP monomers; however, binding by thymosin is preferred over profilin-binding. This affinity for ATP monomers ensures that the pool of unpolymerized actin consists almost entirely of ATP-G-actin with no significant amount of ADP-G-actin in the thymosin-sequestered pool.

Release of ATP-actin monomers from thymosin β4 occurs as part of the mechanism that drives rapid actin polymerization in the normal function of the cytoskeleton in cell morphology and cell motility. Thymosin maintains the bulk of the monomer pool due to its abundance and high affinity for ATP monomers. Maintenance of a high concentration of available monomers at nuclei or filament barbed ends is necessary for rapid actin polymerization. The low affinity of the interaction between ADP monomers and thymosin is necessary to allow monomers to be handed on so that catalysis of nucleotide exchange by profilin regenerates ATP monomers for rapid addition onto filament barbed ends.

Thymosin β4 and profilin serve complementary roles in regulating the polymerization of G-actin. (a) Profilin is bound to PIP2, a membrane lipid, at the cell membrane while the majority of G-actin is bound in a complex with thymosin β4 and thus unable to polymerize. (b) In response to an extracellular signal, such as chemotactic molecules that stimulate actin assembly, profilin is released from the membrane by the hydrolysis of PIP2. The released profilin displaces thymosin β4, forming profilin G-actin complexes that can assemble into filaments. (c) The profilin-actin complexes interact with proline-rich proteins in the membrane, where profilin adds actin monomers to the (+) end of actin filaments. (d) Eventually, the incorporation of monomers into filaments depletes the pools of profilin-actin and thymosin β4 actin complexes. ADP G-actin subunits that have dissociated from a filament are converted into ATP G-actin by profilin, thus helping to replenish the cytoplasmic pool of ATP G-actin.

When thymosin binds to an actin monomer it sterically prevents further binding to and elongation of the plus end of the actin filament. In contrast, when profilin binds to an actin monomer the filament is still capable of elongating. As thymosin and profilin cannot both bind to a single actin monomer at the same time, there is competition between the inhibitory thymosin and the profilin. Generally a majority of actin monomer is thymosin-bound, thus the activation of a small amount of profilin produces rapid filament assembly. Profilin binds to actin monomers that are temporarily released from the thymosin-bound monomer pool, shuttles them onto the plus ends of actin filaments, and is then released and recycled for further rounds of filament elongation.

Therefore, the polymerization of actin is controlled by the give and take between thymosin and profilin. Thymosin basically acts in an inhibitory fashion and interacts with the G-actin monomer and interferes with conformations (ATP and ADP bound forms) to control the assembly of microfilaments.

Skin is the largest organ of the body, which makes up 16% of total body weight. It is also the largest organ that provides immune protection and plays a role in inflammation. Composed of specialized epithelial and connective tissue cells, skin is our major interface with the environment, a shield from the outside world and a means of interacting with it. As such, the skin is subjected to insults and injuries: burns from the sun's ultraviolet radiation that elicit inflammatory reactions, damage from environmental pollutants and wear and tear that comes with aging.

Thymosin beta 4 accelerated skin wound healing in a rat model of a full thickness wound where the epithelial layer was destroyed. When Tβ4 was applied topically to the wound or injected into the animal, epithelial layer restoration in the wound was increased 42% by day four and 61% by day seven, after treatment, compared to untreated. Furthermore, Tβ4 stimulated collagen deposition in the wound and angiogenesis. Tβ4 accelerated keratinocyte migration, resulting in the wound contracting by more than 11%, compared to untreated wounds, to close the skin gap in the wound. An analysis of skin sections (histological observations) showed that the Tβ4-treated wounds healed faster than the untreated. Proof of accelerated cell migration was also seen in vitro, where Tβ4 increased keratinocyte migration two to threefold, within four to five hours after treatment, compared to untreated keratinocytes.

A critical step in wound healing is angiogenesis. New vessels are needed to supply nutrients and oxygen to the cells involved in repair, to remove toxic materials and debris of dead cells and generate optimal conditions for new tissue formation. Another important step is the directional migration of cells into the injured area, joining up to repair the wound. This requires an attractant that will direct the cells to the wound and propel them to the site. These critical steps in wound healing are regulated by Tβ4, as seen in the following experiments

### II. STEM CELLS

Stem cells are primal cells found in all multi-cellular organisms. They retain the ability to renew themselves through mitotic cell division and can differentiate into a diverse range of specialized cell types. Research in the human stem cell field grew out of findings by Canadian scientists Ernest A. McCulloch and James E. Till in the 1960s.

The three broad categories of mammalian stem cells are: embryonic stem cells, derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body, replenishing specialized cells.

As stem cells can be grown and transformed into specialized cells with characteristics consistent with cells of various tissues such as muscles or nerves through cell culture, their use in medical therapies has been proposed. In particular, embryonic cell lines, autologous embryonic stem cells generated through therapeutic cloning, and highly plastic adult stem cells from the umbilical cord blood or bone marrow are touted as promising candidates.

“Potency” specifies the differentiation potential (the potential to differentiate into different cell types) of the stem cell. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal which distinguishes them from non-stem cells.

A. Adult Stem Cells

Adult stem cells, a cell which is found in a developed organism, has two properties: the ability to divide and create another cell like itself, and also divide and create a cell more differentiated than itself. Pluripotent adult stem cells are rare and generally small in number but can be found in a number of tissues including umbilical cord blood. Most adult stem cells are lineage restricted (multipotent) and are generally referred to by their tissue origin (mesenchymal stem cell, adipose-derived stem cell, endothelial stem cell, etc.). A great deal of adult stem cell research has focused on clarifying their capacity to divide or self-renew indefinitely and their differentiation potential.

While embryonic stem cell potential remains untested, adult stem cell treatments have been used for many years to successfully treat leukemia and related bone/blood cancers through bone marrow transplants. The use of adult stem cells in research and therapy is not as controversial as embryonic stem cells, because the production of adult stem cells does not require the destruction of an embryo. Consequently, more U.S. government funding is being provided for adult stem cell research.

The present invention contemplates, in particular, peripheral blood mononuclear cells as a source for cardiogenic stem cells.

Evidence for potential stem cell-based therapies for heart disease has been provided by studies showing that human adult stem cells, taken from the bone marrow, are capable of giving rise to vascular endothelial cells when transplanted into rats. Such stem cells demonstrated plasticity, meaning that they become cell types that they would not normally be. The cells were used to form new blood vessels in the damaged area of the rats' hearts and to encourage proliferation of preexisting vasculature following the experimental heart attack.

Like the mouse stem cells, human hematopoietic stem cells can be induced under the appropriate culture conditions to differentiate into numerous tissue types, including cardiac muscle. When injected into the bloodstream leading to the damaged rat heart, these cells prevented the death of hypertrophied or thickened but otherwise viable myocardial cells and reduced progressive formation of collagen fibers and scars. Furthermore, hematopoietic cells can be identified on the basis of highly specific cell markers that differentiate them from cardiomyocyte precursor cells, enabling such cells to be used alone or in conjunction with myocyte-regeneration strategies or pharmacological therapies.

Table 2, below, lists cell surface markers that can be used to identify cardiogenic stem cells. In particular, Flk1⁺ cells are contemplated.

TABLE 2
Cardiac Progenitor Markers

| Cell Marker | Cell Type | Significance |
|---|---|---|
| MyoD and Pax7 | Myoblast, myocyte | Transcription factors that |
| | | direct differentiation of |
| | | myoblasts into mature |
| | | myocytes |
| Myogenin and MR4 | Skeletal myocyte | Secondary transcription |
| | | factors required for |
| | | differentiation of myoblasts |
| | | from muscle stem cells |
| Myosin heavy chain | Cardiomyocyte | A component of structural and |
| | | contractile protein found in |
| | | cardiomyocyte |
| Myosin light chain | Skeletal myocyte | A component of structural and |
| | | contractile protein found in |
| | | skeletal myocyte |

### III. DETECTION OF CELL SURFACE MARKERS

In accordance with the present invention, one will seek to obtain various stem cell populations by screening of cell populations for appropriate cell surface markers, as discussed above. Generally, this is performed by labeling or physically selecting cells that are bound by antibodies to cell determinants that identify the cells as stem, pluripotent or totipotent stem cells. It is particularly contemplated that antibodies will be of particular use in the various cell seperation techniques described below.

A. Antibody Constructs

Antibodies directed against the various cell surface antigens are readily available from commercial sources. While available from commercial sources, it is also contemplated that monoclonal or polyclonal antibodies for use in the context of the invention may be constructed by a person of ordinary skill.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies are also well known in the art (see, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

B. Antibody Conjugates

The instant invention provides for the use of antibodies against various cell surface antigens which are generally of the monoclonal type, and that may be linked to at least one agent to form an antibody conjugate. It is conventional to link or covalently bind or complex at least one desired molecule or moiety. Such a molecule or moiety may be, but is not limited to a reporter molecule. A reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might employ, by way of example, ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might employ, for example, ²¹¹astatine, ¹⁴carbon, ⁵¹chromium, ³⁶chlorine, ⁵⁷cobalt, ⁵⁸cobalt, copper67, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, ^{99m}technicium and/or ⁹⁰yttrium. ¹²⁵I is often being preferred for use in certain embodiments, and ^{99m}technicium and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugate contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

C. Methods of Conjugation

If desired, the compound of interest may be joined to an antibody via a biologically-releasable bond, such as a selectively-cleavable linker or amino acid sequence. Certain linkers will generally be preferred over other linkers, based on differing pharmacologic characteristics and capabilities. For example, linkers that contain a disulfide bond that is sterically “hindered” are to be preferred, due to their greater stability in vivo, thus preventing release of the moiety prior to binding at the site of action.

Additionally, any other linking/coupling agents and/or mechanisms known to those of skill in the art can be used to combine to components or agents with antibodies of the present invention, such as, for example, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, or combinations thereof.

Cross-linking reagents are used to form molecular bridges that tie together functional groups of two different molecules, e.g., a stablizing and coagulating agent. However, it is contemplated that dimers or multimers of the same analog can be made or that heteromeric complexes comprised of different analogs can be created. To link two different compounds in a step-wise manner, hetero-bifunctional cross-linkers can be used that eliminate unwanted homopolymer formation.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single-chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### IV. CELL SEPERATION TECHNIQUES

Methods of separating cell populations and cellular subsets are well known in the art and may be applied to the cell populations of the present invention. Cells purified in this fashion may then be used for stimulation and cell replacement therapy, such as in tissue regeneration purposes. Embryonic stem cells, as well as stem cells for cardiac and other cell types are believed by the inventors to be useful in accordance with the present invention. Stimulating those stem cells from a quiescent condition with compounds of the present invention should promote differentiation. They may also be treated with particular combinations with previously known growth and differentiation factors and then cultured to expand and/or differentiate. The following description sets forth exemplary methods of separation for stem cells based upon the surface expression of various markers.

A. Fluorescence Activated Cell Sorting (FACS)

FACS facilitates the quantitation and/or separation of subpopulations of cells based upon surface markers. Cells to be sorted are first tagged with a fluorescently labeled antibody or other marker specific ligand. Generally, labeled antibodies and ligands are specific for the expression of a phenotype specific cell surface molecule. The labeled cells are then passed through a laser beam and the fluorescence intensity of each cell determined. The sorter distributes the positive and negative cells into label-plus and label-minus wells at a flow rate of approximately 3000 cells per second.

The use of multiple fluorescent tags exciting at different wavelengths allows for sorting based upon multiple or alternate criteria. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Thus, for example, a single PBMC sample may be analyzed with alternatively labeled anti-Ig antibody, anti-CD3 antibody, anti-CD8 antibody and anti-CD4 antibody to screen for the presence of B cells and T cells within the sample, as well as distinguishing specific T cell subsets.

FACS analysis and cell sorting is carried out on a flow cytometer. A flow cytometer generally consists of a light source, normally a laser, collection optics, electronics and a computer to translate signals to data. Scattered and emitted fluorescent light is collected by two lenses (one positioned in front of the light source and one set at right angles) and by a series of optics, beam splitters and filters, which allow for specific bands of fluorescence to be measured.

Flow cytometer apparatus permit quantitative multiparameter analysis of cellular properties at rates of several thousand cells per second. These instruments provide the ability to differentiate among cell types. Data are often displayed in one-dimensional (histogram) or two-dimensional (contour plot, scatter plot) frequency distributions of measured variables. The partitioning of multiparameter data files involves consecutive use of the interactive one- or two-dimensional graphics programs.

Quantitative analysis of multiparameter flow cytometric data for rapid cell detection consists of two stages: cell class characterization and sample processing. In general, the process of cell class characterization partitions the cell feature into cells of interest and not of interest. Then, in sample processing, each cell is classified in one of the two categories according to the region in which it falls. Analysis of the class of cells is very important, as high detection performance may be expected only if an appropriate characteristic of the cells is obtained.

Not only is cell analysis performed by flow cytometry, but so too is sorting of cells. In U.S. Pat. No. 3,826,364, an apparatus is disclosed which physically separates particles, such as functionally different cell types. In this machine, a laser provides illumination which is focused on the stream of particles by a suitable lens or lens system so that there is highly localized scatter from the particles therein. In addition, high intensity source illumination is directed onto the stream of particles for the excitation of fluorescent particles in the stream. Certain particles in the stream may be selectively charged and then separated by deflecting them into designated receptacles. A classic form of this separation is via fluorescent tagged antibodies, which are used to mark one or more cell types for separation.

Additional and alternate methods for performing flow cytometry and fluorescent antibody cell sorting are described in U.S. Pat. Nos. 4,284,412; 4,989,977; 4,498,766; 5,478,722; 4,857,451; 4,774,189; 4,767,206; 4,714,682; 5,160,974; and 4,661,913, herein expressly incorporated by reference.

B. Micro-Bead Separation

Cells in suspension may be separated to very high purity according to their surface antigens using micro-bead technologies. The basic concept in micro-bead separations is to selectively bind the biomaterial of interest (e.g., a specific cell, protein, or DNA sequence) to a particle and then separate it from its surrounding matrix. Micro-bead separation involves contacting a cell suspension with a slurry of microbeads labeled with a cell surface specific antibody or ligand. Cells labeled with the micro-beads are then separated using an affinity capture method specific for some property of the beads. This format facilitates both positive and negative selection.

Magnetic beads are uniform, superparamagnetic beads generally coated with an affinity tag such as recombinant streptavidin that will bind biotinylated immunoglobulins, or other biotinylated molecules such as, for example, peptides/proteins or lectins. Magnetic beads are generally uniform micro- or nanoparticles of Fe₃O₄. These particles are superparamagnetic, meaning that they are attracted to a magnetic field but retain no residual magnetism after the field is removed. Suspended superparamagnetic particles tagged to a cell of interest can be removed from a matrix using a magnetic field, but they do not agglomerate (i.e., they stay suspended) after removal of the field.

A common format for separations involving superparamagnetic nanoparticles is to disperse the beads within the pores of larger microparticles. These microparticles are coated with a monoclonal antibody for a cell-surface antigen. The antibody-tagged, superparamagnetic microparticles are then introduced into a cellular suspension. The particles bind to cells expressing the surface antigen of interest and maybe separated out with the application of a magnetic field. This may be facilitated by running the suspension over a high gradient magnetic separation column placed in a strong magnetic field. The magnetically labeled cells are retained in the column while non-labeled cells pass through. When the column is removed from the magnetic field, the magnetically retained cells are eluted. Both, labeled and non-labeled fractions can be completely recovered.

C. Affinity Chromatography

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed elsewhere in this document.

### V. STEM/PROGENITOR/DIFFERENTIATED CELL CULTURE

Cell culture facilitates the maintenance and propagation of cells in vitro under controlled conditions. Cells may be cultured in a variety of types of vessels constructed of, for example, glass or plastic. The surfaces of culture vessels may be pre-treated or coated with, for example, collagen, polylysine, or components of the extracellular matrix, to facilitate the cellular adherence. Some sophisticated techniques utilize entire layers of adherent cells, feeder cells, which are used to support the growth of cells with more demanding growth requirements.

Cells are normally cultured under conditions designed to closely mimic those observed in vivo. In order to mimic the normal physiological environment cells are generally incubated in a CO₂ atmosphere with semi-synthetic growth media. Culture media is buffered and contains, among other things, amino acids, nucleotides, salts, vitamins, and also a supplement of serum such as fetal calf serum (FCS) horse serum or even human serum. Culture media may be further supplemented with growth factors and inhibitors such as hormones, transferrin, insulin, selenium, and attachment factors.

As a rule, cells grown in vitro do not organize themselves into tissues. Instead, cultured cells grow as monolayers (or in some instances as multilayers) on the surface of tissue culture dishes. The cells usually multiply until they come into contact with each other to form a monolayer and stop growing when they come into contact with each other due to contact inhibition.

Anchorage-dependent cells show the phenomenon of adherence, i.e., they grow and multiply only if attached to the inert surface of a culture dish or another suitable support. Such cells cannot normally be grown without a solid support. Many cells do not require this solid surface and show a phenomenon known as Anchorage-independent growth. Accordingly, one variant of growing these cells in culture is the use of Spinner cultures or suspension cultures in which single cells float freely in the medium and are maintained in suspension by constant stirring or agitation. This technique is particularly useful for growing large amounts of cells in batch cultures.

Anchorage-independent cells are usually capable of forming colonies in semisolid media. Some techniques have been developed that can be used also to grow anchorage-dependent cells in spinner cultures. They make use of microscopically small positively-charged dextran beads to which these cells can attach.

The starting material for the establishment of a cell culture typically is tissue from a suitable donor obtained under sterile conditions. The tissues may be minced and treated with proteolytic enzymes such as trypsin, collagenase of dispase to obtain a single cell suspension that can be used to inoculate a culture dish. In some cases dispersion of tissue is also effectively achieved by treatment with buffers containing EDTA. A particular form of initiating a cell culture is the use of tiny pieces of tissues from which cells may grow out in vitro.

Primary cell cultures maintained for several passages may undergo a crisis. Ascrisis is usually associated with alterations of the properties of the cells and may proceed quickly or extend over many passages. Loss of contact inhibition is frequently an indication of cells having lost their normal characteristics. These cells then grow as multilayers in tissue culture dishes. The most pronounced feature of abnormal cells is the alteration in chromosome numbers, with many cells surviving this process being aneuploid. The switch to abnormal chromosome numbers is usually referred to as cell transformation and this process may give rise to cells that can then be cultivated for indefinite periods of time by serial passaging. Transformed cells give rise to continuous cell lines.

In certain aspects of the instant invention, cells are cultured prior to contact with differentiating agents. They may also be cultured after contact, i.e., after they have been induced to differentiate toward a given or specific phenotype. Cells will be cultured under specified conditions to achieve particular types of differentiation, and provided various factors necessary to facilitate the desired differentiation.

### VI. STIMULATORY FACTORS

A. Cell Growth and Differentiation Factors

Cell growth and differentiation factors are molecules that stimulate cells to proliferate and/or promote differentiation of cell types into functionally mature forms. In some embodiments of the invention, cell growth and differentiation factors may be administered in combination with compounds of the present invention in order to direct the administered cells to proliferate and differentiate in a specific manner. One of ordinary skill would recognize that the various factors may be administered prior to, concurrently with, or subsequent to the administration of compounds of the present invention. In addition, administration of the growth and/or differentiation factors may be repeated as needed.

It is envisioned that a growth and/or differentiation factor may constitute a hormone, cytokine, hematapoietin, colony stimulating factor, interleukin, interferon, growth factor, other endocrine factor or combination thereof that act as intercellular mediators. Examples of such intercellular mediators are lymphokines, monokines, growth factors and traditional polypeptide hormones. Included among the growth factors are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factors-α and -β.; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte/macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; IL-13, IL-14, IL-15, IL-16, IL-17, IL-18. As used herein, the term growth and/or differentiation factors include proteins from natural sources or from recombinant cell culture and biologically active equivalents of the native sequence, including synthetic molecules and mimetics.

B. Post-Stimulation Purification of Induced Cells

Following stimulation, it may be desirable to isolate stem cells that have been induced to undergo differentiation from those that have not. As discuss above, a variety of purification procedures may be applied to cells to effect their separation, and a number of these rely on cell surface markers.

U.S. Patent Publication No. 2005/0164382, incorporated herein by reference, describes methods of obtaining cardiomyoctyes as well as various cardiomyocyte markers including cTnI, cTNT, ventricular myosin, connexin43, sarcomeric myosin heavy chain (MHC), GATA-4, Nkx2.5, N-cadherin, P1-adrenoceptor (β1-AR), ANF, MEF-2A, MEF-2B MEF-2C, MEF-2D creatine kinase MB (CK-MB), myoglobin, or atrial natriuretic factor (ANF).

### VII. METHODS OF TREATMENT

The present invention contemplates a variety of uses for the compounds of the present invention. In particular, they can be used to treat individuals that have undergone trauma, injury, disease or other destruction or damage to cardiac tissue. In particular, the invention is directed to the treatment of damaged heart muscle in the context of cardiac hypertrophy, myocardial ischemia and cardiac failure.

In one embodiment, the invention contemplates the administration of the compounds directly into an affected subject. Traditional routes and modes of administration may be utilized depending the clinical situation and the tissue target of the therapy. Alternatively, the invention may rely on an ex vivo approach, where stem cells are stimulated with compounds of the present invention outside an organism and then administered, optionally after culturing to expand the cells, to a recipient. The cells may be heterologous to the recipient, or they may have previously been obtained from that recipient, i.e., autologous.

### VIII. PHARMACEUTICAL COMPOSITIONS

It is envisioned that, for administration to a host, compounds of the present invention and stimulated/differentiated cells will be suspended in a formulation suitable for administration to a host. Aqueous compositions of the present invention comprise an effective amount of a compound and/or cells dispersed in a pharmaceutically acceptable formulation and/or aqueous medium. The phrases “pharmaceutically and/or pharmacologically acceptable” refer to compositions that do not produce an adverse, allergic and/or other untoward reaction when administered to an animal, and specifically to humans, as appropriate.

As used herein, “pharmaceutically acceptable carrier” includes any solvents, dispersion media, coatings, antibacterial and/or antifungal agents, isotonic and/or absorption delaying agents and the like. The use of such media or agents for pharmaceutical active substances is well known in the art. Except insofar as any conventional media or agent is incompatible with the active ingredient, its use in the therapeutic compositions is contemplated. Supplementary active ingredients can also be incorporated into the compositions. For administration to humans, preparations should meet sterility, pyrogenicity, general safety and/or purity standards as required by FDA Office of Biologics standards.

Compounds and/or cells for administration will generally be formulated for parenteral administration, e.g., formulated for injection via the intravenous, intramuscular, sub-cutaneous, intralesional, or even intraperitoneal routes. The preparation of an aqueous composition that contains cells as a viable component or ingredient will be known to those of skill in the art in light of the present disclosure. In all cases the form should be sterile and must be fluid to the extent that easy syringability exists and that viability of the cells is maintained. It is generally contemplated that the majority of culture media will be removed from cells prior to administration.

Generally, dispersions are prepared by incorporating the compounds and cells into a sterile vehicle which contains the basic dispersion medium and the required other ingredients for maintaining cell viability as well as potentially additional components to effect proliferation, differentiation or replacment/grafting in vivo. Upon formulation, solutions will be administered in a manner compatible with the dosage formulation or in such amount as is therapeutically effective. Some variation in dosage will necessarily occur depending on the condition of the subject being treated. The person responsible for administration will, in any event, determine the appropriate dose for the individual subject.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Method

Peptide synthesis. Peptides were synthesized on Wang resins (Novabiochem) using an automated Applied Biosystems (Life Technologies) 433A synthesizer. All amino acids and resins were purchased from Novabiochem, and all other reagents and solvents were obtained from Fisher, Novabiochem and Aldrich. Highly optimized fluorenylmethoxycarbonyl (Fmoc) chemical protocols, based on previously described procedures (Ball and Mascagni, 1996), with 2-(1H-benzotriazol-1yl)-1,1,3,3-tetramethyluronium hexafluorophosphate and N-hydroxybenzotriazole activation were used. For the 0.25 mmole scale syntheses a capping procedure was performed with N-(2-chlorobenzyloxycarbonyloxy)succinimide (Ball and Mascagni, 1997), otherwise 0.1 mmole scale syntheses were used without capping. Deprotection of the peptide and cleavage from the resin was achieved using 95% TFA containing the scavengers ethanedithiol and thioanisole (1:2). The cleavage reaction was performed at room temperature for 1.5-3 hours depending on the length of the peptide. The cleaved peptide was precipitated in diethyl ether, centrifuged and washed several times with fresh ether. Purification of the crude peptides was performed on either C4 or C18 Grace-Vydac (Hisperia, Calif.) semi-preparative reversed-phase high-pressure liquid chromatography columns (250×10 mm) using a Waters 600 HPLC system. Fractions were analyzed on a Vydac C18 analytical column (150×4.6 mm). Separations were achieved using linear gradients of 0 to 100% buffer B for 180 or 30 min, at a flow rate of either 3 or 1 ml/min, respectively. Buffer A was water-0.045% TFA, and buffer B was acetonitrile-0.036% TFA. Detection was at 220 nm. The identity of the peptides was confirmed using either MALDI-TOF (Waters LR MALDI-TOF) or ESI-QTOF MS (ABI-Sciex QStar XL).

In vitro embryonic cardiac cell migration assay. The outflow tract of the heart was dissected from E11.5 wild-type mouse embryos and placed on rat-tail collagen matrix (Roche) endothelial layer facing down as described (Bock-Marquette et al., 2004; Runyan and Markwald, 1983). After 10 h of attachment, three independent explants were incubated with 300 ng of peptide #1-17, 100 ng of TB4, or 3 μl PBS in 300 ml OPTIMEM medium (Invitrogen/Gibco) respectively. Medium and protein were changed every two days. To monitor migration distance photos were taken daily using Nikon DXM1200F microscope at 4× magnification. Cell migration distance was quantified by ImageJ software (http at rsb.info.nih.gov/ij/) in three separate explants and three measurements for each treatment respectively. Beating frequency of myocardial cells was counted three times at 7-15 days of treatment. Calculations were normalized to PBS and TB4 treated positive and negative controls. After 15 days at 37° C. in 5% CO₂, explants were fixed in 4% paraformaldehyde in PBS for 10 min at room temperature. Explants were stored at 4° C. in PBS for further immunocytochemical investigations.

Animals and Surgical procedures. Mice: Myocardial infarctions were produced in seventy C57BL/6J male mice at 16 weeks of age (25-30 g) by ligation of the left anterior descending (LAD) coronary artery as described (Garner et al., 2003). All animal protocols were reviewed and approved by the University of Texas Southwestern Medical School Institutional Animal Care Advisory Committee and were in compliance with the rules governing animal use as published by the NIH. Mice were sedated in an isoflurane chamber (5%) for 60 seconds until self-designed coaxial mask could be safely applied. The mask supplied continuous isoflurane (2.0%) and oxygen (98.0%) under positive pressure from a Harvard small animal respirator throughout the procedures. Immediately after ligation mice were injected with 200 ng of peptide in 4.4 ml collagen or 4.4 ml PBS in collagen intracardially in the infarction border, and with 150 mg peptide in 300 ml of PBS or 300 ml PBS intraperitoneally as described (Bock-Marquette et al., 2004). Intraperitoneal injections were repeated every three days after ligation. Effective doses of the peptides were calculated based on optimal in vitro cell migration effect and on biodistribution calculations of full length TB4 (Mora et al., 1997). The inventors administered buprenorphine (0.05 mg/kg) for post-operative pain control. Hearts were removed 1, 14 and 21 days after ligation and processed for further investigations.

Analysis of cardiac function by echocardiography: Echocardiograms to assess systolic function were performed using M-mode and 2-dimensional measurements as described previously (Garner et al., 2003). The measurements represented the average of six selected cardiac cycles from at least two separate scans performed in random-blind fashion with papillary muscles used as a point of reference for consistency in level of scan. End diastole was defined as the maximal left ventricle (LV) diastolic dimension and end systole was defined as the peak of posterior wall motion. Single outliers in each group were omitted for statistical analysis. Fractional shortening (FS), a surrogate of systolic function, was calculated from LV dimensions as follows: FS=[(EDD−ESD)/EDD]×100%. Ejection fraction (EF) was calculated as follows: EF=[(edd³−esd³)/edd³]×100%. EDD, end diastolic dimension; ESD, end systolic dimension.

Calculation of scar volume and myocyte size: Scar volume was calculated after Masson's trichrome staining in six sections through the heart of each mouse using Openlab 3.03 software (Improvision) similar to previously described (Bock-Marquette et al., 2004). Percent area of collagen deposition was measured on each section in blinded fashion and averaged for each mouse.

Pigs: German pigs were purchased from the animal research department of the Ludwig-Maximilians-University (Oberschleissheim, Germany). Animal care and all experimental procedures were performed in accordance to the German and NIH animal legislation guidelines and were approved by the Bavarian Animal Care and Use Committee (AZ 55.2-1-54-2531-26/09). All animal experiments were conducted at the Walter Brendel Centre of Experimental Medicine, University of Munich.

Ischemia reperfusion experiments: Pigs (n=5/experimental group) were anesthetized and instrumented as described previously (Hinkel et al., 2008). Briefly, a balloon was placed in the left anterior descending artery (LAD) distal to the bifurcation of the first diagonal branch and inflated for 60 min. Correct localization of the coronary occlusion and patency of the first diagonal branch were ensured by injection fluoroscopy. Regional application of TB4 peptides or saline solution was performed via selective pressure regulated retroinfusion for 10 min into the anterior interventricular vein (AIV) draining the LAD-perfused myocardium (Kupatt et al., 2005).

Global myocardial function: At day 0 before ischemia induction and after 24 h of reperfusion myocardial function was obtained via Millar pressure tip catheter. Therefore the Millar pressure tip catheter was placed in the left ventricle and left ventricular pressure was recorded for off-line analysis. ECG trigger analysis was performed for left ventricular end-diastolic pressure, displayed as mmHg for the different time points.

Regional myocardial function: 24 hours after induction of ischemia, sternotomy was performed and ultrasonic crystals were placed subendocardial into the area at risk, the infracted area as well as into the Cx perfusion area in a standardized manner. Subendocardial segment shortening (SES) was assessed under resting heart rate as well as at 120 and 150 beats/min atrial pacing (for 1 min/each).

Infarct size and local inflammation: For assessment of infarct size methylene blue was injected in the left ventricle for negative staining of the AAR and tetrazolium red was applied into the LAD for viability staining in the infracted area. Thereafter, left ventricle was cut into slices (5 mm thick), and subjected to digital photography. Planimetry of different slices was conducted via ImageJ for assessment of infarct size in % of AAR. Tissue samples from infarct, area at risk and non-ischemic control were harvested for leukocyte recruitment. Myeloperoxidase assays were performed as previously described (Kupatt et al., 2005).

AGES Biodistribution studies. In vitro: 600 nmoles of AGES peptide was conjugated using 6 nmoles of mono-sulfo-hydroxi-succimido nanogold (Nanoprobes, Yaphank, N.Y.) as recommended by the manufacturer. Unconjugated nanogold paricles were separated by gel filtration (Bio-Gel P-10, Bio-Rad, Hercules Calif.). 50 ml of labeled purified peptide-conjugate in PBS was added to adult human cardiomyocytes or adult human coronary endothelial cells (PromoCell, Germany) on fibronectin coated cover slips in 350 ml medium. Cells were incubated for 5, 10, 20, 30, 40 and 50 minutes at 37° C. and fixed in 5% Glutaraldehyde in PBS for 10 minutes. To visualize gold particles, silver enhancement was applied for 20 minutes as recommended by the manufacturer (Nanoprobes Yaphank, N.Y.). Sarcomeric α-actinin (1:200) (Sigma) and p-Cytokeratin (1:50) (Santa Cruz) primary antibodies were used to indicate cell types.

In vivo: Small animal imaging studies were performed using Siemens Inveon PET-CT Multimodality system (Siemens Medical Solutions Inc., Knoxville, Tenn., USA). Ten minutes prior imaging animals were anesthetized using 3% Isofluorane until stable vitals were established. After sedation, animals were kept under 2% Isofluorane anesthesia for the duration of the procedures. CT imaging was acquired at 80 kV and 500 mA with a focal spot of 58 mm. Total rotation of the gantry was 360° with 360 rotation steps obtained at 235 ms/frame exposure time. Images were attained using CCD readout of 4096×3098 with a bin factor of 4 and an average frame of 1. Effective pixel size was 103.03 mm under low magnification. Total scan time was approximately 6 min. Images were reconstructed with a down sample factor of 2 using Cobra Reconstruction Software. PET imaging was acquired directly after acquisition of CT data. Radiotracer (⁶⁸Ga-AGES (n=3) or ⁶⁸Ga-Control (n=3)) was injected intravenously via the tail vein. A dynamic PET scan from 0-60 min post-injection (p.i.) was performed on each animal. PET images were reconstructed using Fourier Rebinning and Ordered Subsets Expectation Maximization 3D (OSEM3D) algorithm. Reconstructed images were fused and analyzed using Inveon Research Workplace (IRW) software. For quantification, regions of interest were placed in the areas expressing the highest radiotracer activity as determined by visual inspection. Tissues examined include the heart, liver, lung, brain, kidneys, bladder and skeletal muscle. Quantitative data were expressed in Percent Injected Dose per Gram (% ID/g).

Western blot. One, fourteen and twenty-one days after systemic injection of peptides, TB4 or PBS (n=3/each treatment), mice were sedated. Hearts were perfused with saline to remove blood. The intact area and infarcted core of the hearts were separated immediately frozen in liquid nitrogen. Samples were placed in 1 ml of Trizol reagent directly before homogenization (Invitrogen, Carslbad, Calif.). The protein fraction was isolated from the interphase of the Trizol purification as recommended by the manufacturer; 10 μg of the total protein was loaded and analyzed by Western blot with protein-specific primary antibodies: VEGF, p-Marcks, p-Akt, and GAPDH (1:100) (Santa Cruz), Connexin43 (1:50) (Invitrogen), N-Cadherin (1:5000) (BD Biosciences) and pS368-Connexin43 (1:1000) (Cell Signaling Technology).

Immunohistochemistry. Adult mouse cardiac tissue was fixed in 4% paraformaldehyde and sections were analyzed by immunohistochemistry. Hearts were sectioned at eight levels from the base to the apex. Serial sections were immunostained with primary antibodies against Pecam-1 and Wt-1 (1:50) (Santa Cruz), Smooth muscle a-actin (1:220) (Sigma), Sarcomeric a-actinin (1:200) (Sigma), Connexin43 (1:200) (Invitrogen), pS368-Connexin43 (1:200) (Cell Signaling Technology) as recommended by the manufacturer. Terminal deoxynucleotidyl transferase dUTP nick end labeling (TUNEL) assay was performed using In Situ Cell Detection kit (Roche). Number of sm a-actin positive capillaries and Wt-1 positive cells were counted at the infarction border zone in high power photographs at all levels of the sections in three peptide, TB4 and PBS treated hearts respectively.

Statistics. Statistical calculations were performed using a standard t-test of variables with 95% confidence intervals. Differences between groups were considered significant for p<0.05. Statistical analysis specific for pigs was done using one-way analysis of variance (ANOVA). Whenever a significant effect was obtained with ANOVA, the inventors used multiple comparison tests between the groups with the Student-Newman-Keul's procedure (SPSS 17.0 statistical program). Differences between groups were considered significant for p<0.05. The results are given as mean values +/−SEM.

Chemistry. The bifunctional chelator 1 was synthesized according to previously reported procedure (Eisenwiener et al., 2002). The terminal carboxylic acid of 1 was activated by N-hydroxysuccinimide (NHS) and conjugated to the peptide in the presence of N,N-diisopropylethylamine (DIPEA). The peptide-conjugate 2 was deprotected using 95% trifluoroacetic acid (TFA) to give 3 quantitative yield. The peptide-conjugate H₃3 was purified by HPLC and lyophilized. Conjugates 2 and H₃3 were characterized by Matrix Assisted Laser Desorption/Ionization Time-of-Flight (MALDI-TOF) Mass Spectra. Purity was assured by a single peak on reverse-phase HPLC. Control H₃4 was prepared by deprotecting 1 with 95% TFA in quantitative yield. Radiolabeling of H₃3 with ⁶⁸Ga was performed at 70° C. for 30 min and confirmed by radio-TLC developed in 10% NH₄OAc and methanol (1:4 v/v). ⁶⁸Ga-labeled peptide conjugate (⁶⁸Ga-3) was purified by reverse-phase HPLC (t=8.4 min). The corrected radiochemical yield was >91%. Chemical identity of the purified compound was confirmed by matching its elution profile with cold Ga-3. Compound H₃4 was radiolabeled using 0.5 N NaOH to adjust pH to 3. After completion of the reaction solution was neutralized to pH 7.

General Methods and Materials. All reactions were carried out under an argon atmosphere in degassed dried solvents. Commercially available starting compounds were purchased from commercial vendors and used directly without further purification unless otherwise noted. Analytical thin-layer chromatography (TLC) was performed using Merck 60 F254 silica gel (precoated sheets, 0.2 mm thick) (Lawrence, Kans.). The spectra of ¹H NMR were recorded on a Varian 400 or 500 MHz spectrometer. (MALDI) mass spectra were acquired on an Applied Biosystems Voyager-6115 mass spectrometer. Bulk solvent removal was done by rotary evaporation under reduced pressure, and trace solvent was removed by vacuum pump. Compound 1 was synthesized according to the published procedure (Eisenwiener et al., 2002). The ^{69/71}GaCl₃ was purchased from Sigma Aldrich. ⁶⁸GaCl₃ was produced in an in-house ⁶⁸Ge/⁶⁸Ga generator system purchased from (iThemba LABS (Somerset west, South Africa)).

HPLC Methods. High performance liquid chromatography (HPLC) was performed on a Waters 600 Multisolvent Delivery System equipped with a Waters 2996 Photodiode Array detector. The mobile phase was H₂O with 0.1% TFA (solvent A) and acetonitrile with 0.1% TFA (solvent B). The analytical analysis and purification was performed on an XTerra RP18 Column with a gradient of 0% B to 100% B in 50 min at a flow rate of 4.0 mL/min.

Compound H₃3. To a mixture of compound 1 (5.0 mg, 9.2 μmol), N-hydroxysuccinimide (6.2 mg, 54.0 μmol) and EDC (1-ethyl-3-(3-dimethylaminopropyl)carbodiimide)) (10.5 mg, 54.0 μmol) in 500 μL of dry MeCN was added 20 μL of N, N-diisopropylethylamine (DIPEA). The resulting mixture was then stirred under N₂ for 24 hours. After removal of the solvent under reduced pressure, the residue was redissolved in CHCl₃ (1 ml) and then washed with water (3×2 ml) promptly. The solvent was evaporated, and the residue was dried by a lyophilizer to yield NHS-activated 1 as pale yellow solid (MALDI-TOF/MS: 641.42, M+H⁺). The activated ester was used directly for the next reaction with the peptide [AGES] (5.0 mg, 13.8 μmol) in 500 μL of anhydrous DMF, to which 50 μL of DIPEA was added. The mixture was then stirred at room temperature (r. t.) for 24 hours under N₂. After evaporation of the solvent under vacuum, the crude product was purified by semi-preparative reverse-phase HPLC. The collected fractions of multiple runs were combined and lyophilized to afford 4.5 mg of 2 as white powder (55%). MALDI-TOF/MS: 888.57 [M+H⁺]. The protected conjugate 2 (4.0 mg, 4.5 μmol) was dissolved in 95% of TFA and stirred at r. t. for 12 h. After evaporation of the solvent, the residue was purified by semi-preparative reverse-phase HPLC. The collected fractions of multiple runs were pooled and lyophilized to afford 3.0 mg of H₃3 as white powder (91%). MALDI-TOF/MS: 720.28 [M+H⁺].

Compound H₃4. The bifunctional chelator 1 (5.0 mg, 9.2 μmol) was dissolved in 95% of TFA and stirred at r. t. for 12 h. After evaporation of the solvent, the residue was purified by semi-preparative reverse-phase HPLC. The collected fractions of multiple runs were pooled and lyophilized to afford 3.3 mg of 4 as white powder (87%). MALDI-TOF/MS: 376.17 [M+H⁺].

Cold ^{69/71}Ga-3. To a 1.5 ml vial containing of H₃3 (2 mg, 2.7 μmol) in water (0.5 mL) was added GaCl₃ (4.8 mg, 27 mmol). The reaction mixture was shaken and incubated at 70° C. for 24 h. After evaporation of the solvent under vacuum, the crude product was purified by semi-preparative reverse-phase HPLC. The collected fractions of multiple runs were combined and lyophilized to afford 1.6 mg of ^{69/71}Ga-3 as white powder (73%). MALDI-TOF/MS: 808.06 [M+H₂O⁺]. Radiolabeling of H₃3 with ⁶⁸Ga. To a 1.5 ml vial containing 10 mg of H₃3 in 1400 ml of 1 M HEPES (4-(2-hydroxyethyl)-1-piperazineethanesulfonic acid) solution, 1-1.5 mCi of ⁶⁸GaCl₃ in 0.6 N HCl was added. The reaction mixture was shaken and incubated at 70° C. for 0.5 h. Then, 2 ml of 5 mM ethylenediaminetetraacetic acid (EDTA) was added to the reaction mixture to remove non-specifically bound or free ⁶⁸Ga from the ⁶⁸Ga-labeled conjugate. The ⁶⁸Ga-3 was first analyzed by radio-TLC and purified by reverse-phase HPLC (t=8.4 min). The fraction containing the highest activity was collected and evaporated to give the final compound ⁶⁸Ga-3.

Radiolabeling of H₃4 with ⁶⁸Ga. The pH of 750 ml of ⁶⁸GaCl₃ (1-1.5 mCi) in 0.6 N HCl was adjusted to 3 by adding 500 ml of 0.5 N NaOH. 10 mg of H₃4 was added to this solution, and the reaction mixture was shaken and incubated at 70° C. for 0.5 h. The ⁶⁸Ga-4 was first analyzed by radio-TLC and purified by reverse-phase HPLC (t=4.4 min).

### Example 2

### Results

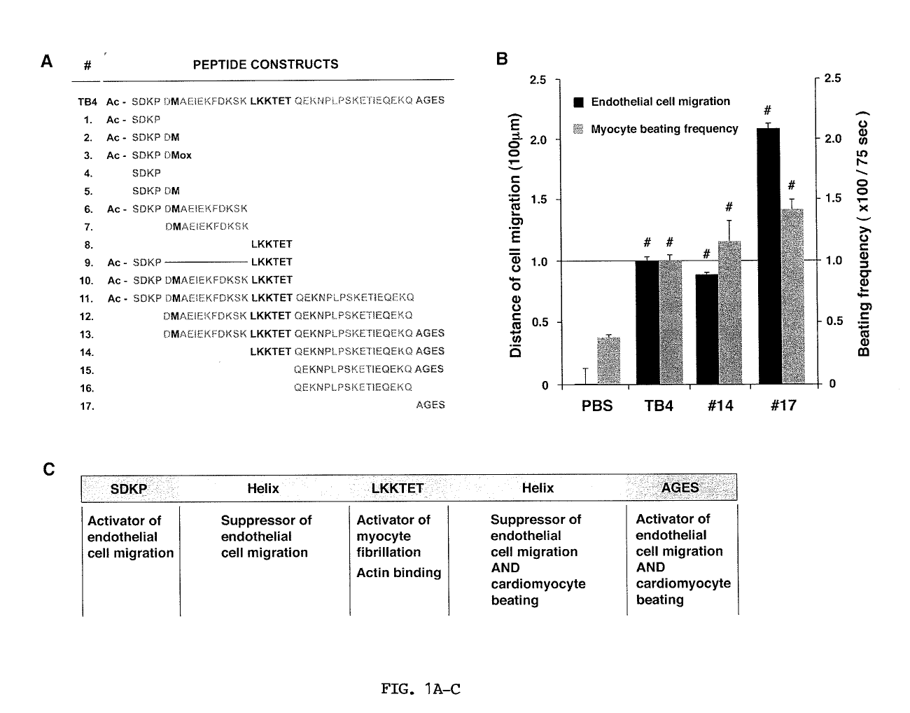

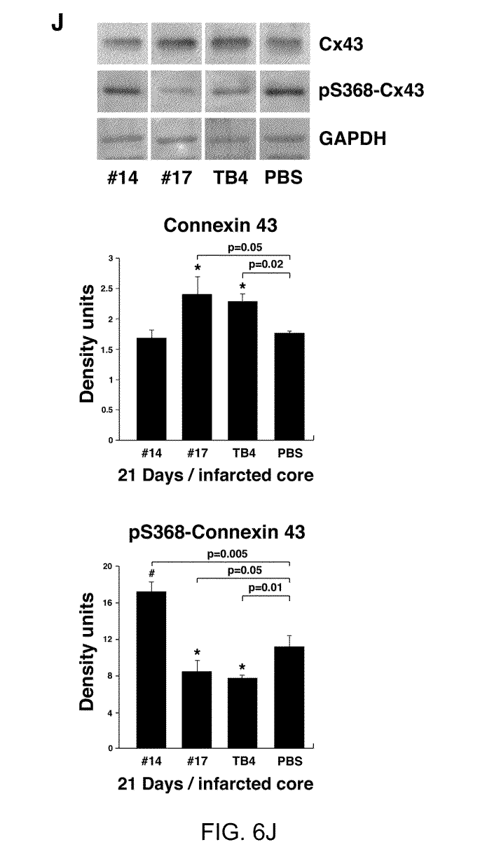

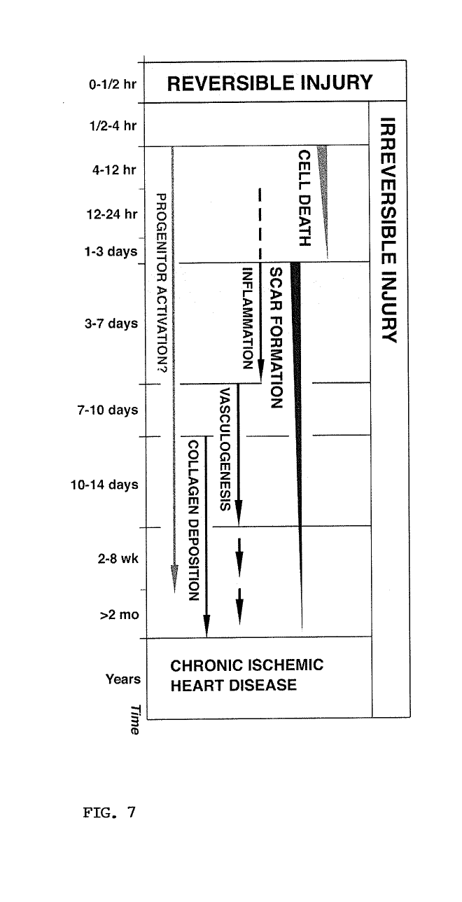

Effect of TB4 domains on embryonic cardiac endothelial cell migration and myocyte beating in vitro. Previous observations suggest, that small changes in TB4 structure cause alterations in its biological function in vitro and in vivo (Hertzog et al., 2004; Huff et al., 1995; Grillon et al., 1990; Rieger et al., 1993; Smart et al., 2007; Liu et al., 2003; Yang et al., 2004; Sosne et al., 2010). No systematic analysis was performed, however, to examine the role of individual TB4 domains in the content of cardiac cell migration, survival and heart repair. Thus, the inventors synthesized domain fragments, or biochemically modified forms of TB4 and tested their physiological impact using embryonic cardiac explants (FIGS. 1A-C, FIG. 7).

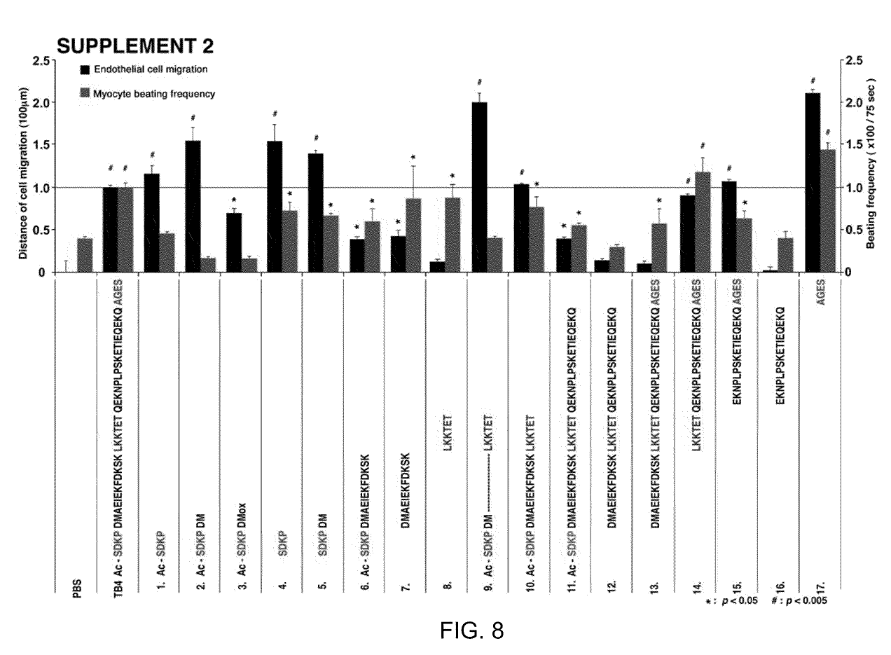

First, the inventors investigated the effect of acetylated and non-acetylated forms of the N-terminal variable domain with or without addition of Met6 (FIG. 1A, FIG. 8 #1-5). Migration results were normalized to PBS control. In agreement with previous findings, Ac-SDKP (#1) significantly increased endothelial cell migration (116.19+/−1.02 mm) similar to full length TB4 (100.00+/−2.38 mm). Ac-SDKP, however, failed to increase the frequency of myocardial beating (45.08+/−2.31 beats/75 sec) when compared to TB4 (100.00+/−5.03 beats/75 sec) (FIG. 8), which may explain why in vivo administration of Ac-SDKP does not aid post-ischemic cardiac function despite its positive influence on fibrosis, inflammation and coronary vessel growth (Yang et al., 2004). Addition of Met6 to the construct further suppressed beating (FIG. 8 #2 10.65+/−1.15 beats/75 sec), while de-acetylation of both peptides increased migration and beating, respectively (FIG. 8 #4 154.07+/−2.02 mm; 86.88+/−1.15 beats/75 sec and #5 139.02+/−4.25 mm; 66.39+/−3.46 beats/75 sec). Oxidation of Met6 residue suppressed both functions (FIG. 8 #3 70.73+/−1.56 mm; 17.21+/−1.00 beats/75 sec).

Next, the inventors investigated the role of the N- (#7) and C-terminal (#16) helixes on cardiac cell behavior. Comparison of peptides #1 and #6 suggest addition of the N-terminal helix inhibits cell migration and slightly increases myocyte beating (from #1 116.19+/−1.02 mm to #6 38.31+/−3.06 mm and from #1 45.08+/−2.31 beats/75 sec to #6 59.40+/−15.01 beats/75 sec). This was also observed when constructs #9 and #10 were compared (from #9 200.17+/−1.09 mm to #10 102.81+/−1.87 mm and from #9 40.16+/−1.15 beats/75 sec to #10 76.58+/−12.51 beats/75 sec). Analyzing the effects of the C-terminal helix (#16), the inventors found it suppresses cell migration and beating respectively (from #10 102.81+/−1.87 mm to #11 40.63+/−0.48 mm and from #10 76.58+/−12.51 beats/75 sec to #11 55.41+/−0.62 beats/75 sec).

Addition of the central actin-binding domain LKKTET (#8) to the medium resulted in increased cell beating without significantly affecting migration when compared to full length TB4 (#8 13.00+/−2.81 mm; 87.02+/−16.17 beats/75 sec). The force and frequency of contractions resembled myocyte fibrillation, suggesting LKKTET may affect conduction and cell-cell communication in vitro.

Finally, the inventors investigated the variable N-terminal region of full length TB4 (#17). They found the short tetrapeptide AGES increases cardiac cell migration and myocyte beating to a greater extent than full length TB4 (#17 211.28+/−2.78 mm; 144.26+/−8.08 beats/75 sec, FIGS. 1A-C). Moreover, conjugation of AGES to other constructs stimulated cell migration and beating (from #16 14.21+/−3.68 mm to #15 105.88+/−3.37 mm and from #16 39.39+/−8.08 beats/75 sec to #15 62.42+/−9.45 beats/75 sec).

In summary, these in vitro results suggest domains of TB4 affect endocardial and myocardial cell behavior distinctly (FIG. 1C). While the N-terminus of TB4 alters primarily endothelial cell migration, the C-terminal domain influences migration and myocyte contraction, respectively. Absence of both variable domains of TB4 results in loss of these effects (FIG. 8 comparisons of TB4 and #12). This may also due to the inhibitory effect of the exposed helices (FIG. 8 comparisons of #8 and #12, #1 and #6, #17 and #15). Since peptide #1 did not affect post-ischemic cardiac function regardless of its benefits on endothelial cell migration and fibrosis (Yang et al., 2004), the inventors predict positive influence on myocyte beating could be the discriminating factor for therapeutic candidate selection. Thus, constructs #14 (close to full length TB4) and #17 (better than full length TB4) became subjects of our further in vivo investigations (FIG. 1B). Construct #3 and PBS served as a negative controls.

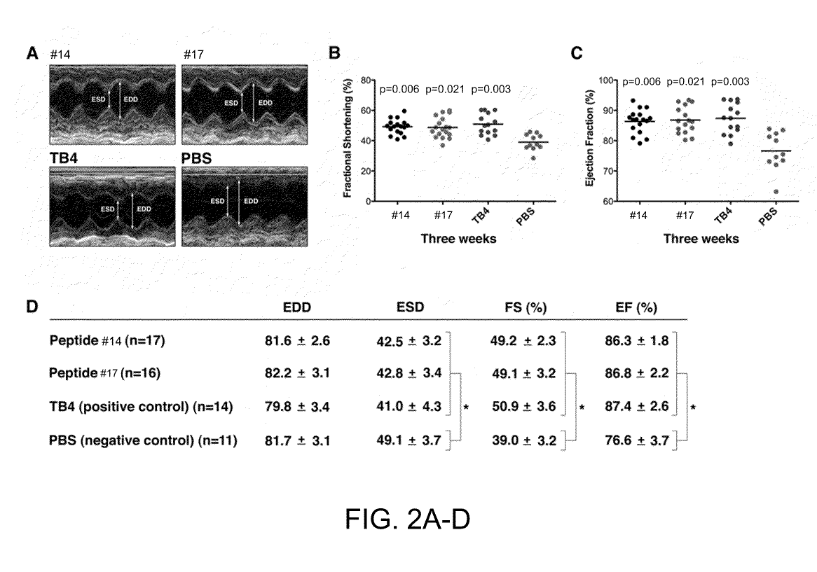

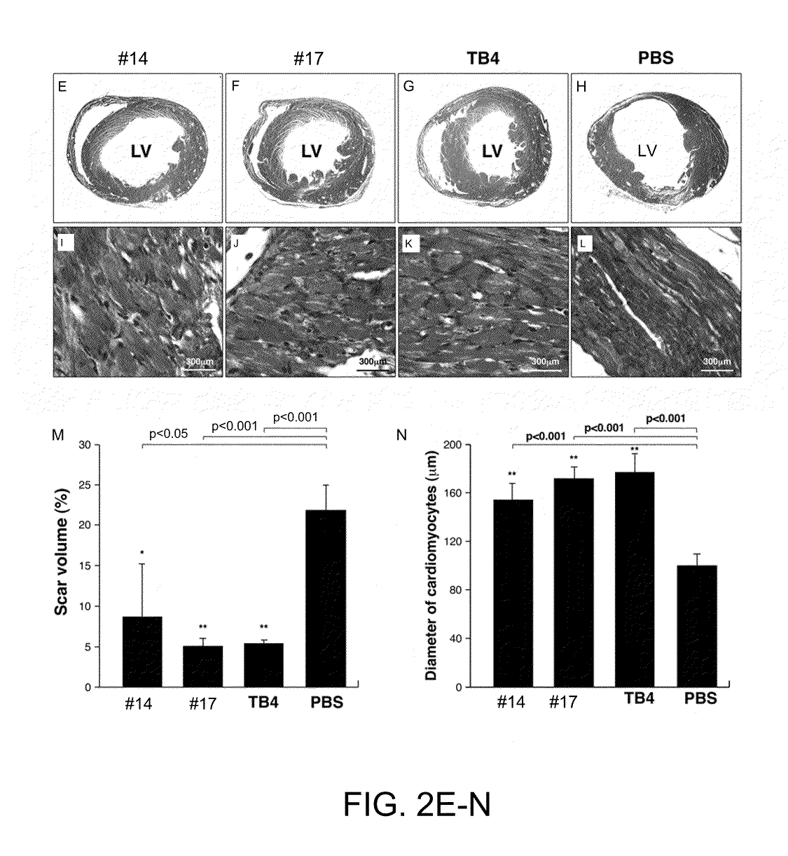

Influence of LKKTETQEKNPLPSKETIEQEKQAGES (#14) and AGES (#17) on post-ischemic cardiac function in adult mice and pigs. To assess the effects of #14 and #17 on cardiac repair in vivo, the inventors ligated the left anterior descending coronary artery (LAD) of 70 adult mice and injected 20 animals with peptides #14 or #17 in PBS and 15 animals with full-length TB4 in PBS or PBS only as controls (FIG. 2A-N). Intracardiac injections were given once directly after ligation, followed by systemic intraperitoneal injections every three days as previously described (Bock-Marquette et al., 2004). The studies were performed and analyzed in blinded fashion, with multiple measurements, and completed within no more than 10 minutes after anesthetic was administered. Twelve animals died during or directly after ligation due to surgical complications. All mice that survived were interrogated for cardiac function by random-blind ultrasonography at one, two and three weeks after infarction by multiple measurements of cardiac contraction. Three weeks after ligation, the mean fractional shortening (FS) was 49.2+/−2.3% (n=17) in #14 treated and 49.1+/−3.2% (n=16) in #17 treated animals. Also, the calculated ejection fraction (EF) was 86.3+/−1.8% after #14 and 86.8+/−2.2% after #17 treatment, which was similar to TB4 injected positive controls (EF: 50.9+/−3.6%; FS: 87.4+/−2.6% (n=14)). Mice with PBS administration showed decreased mean FS of 39.0+/−3.2% and EF of 76.6+/−3.7% (n=11). Analyzing the left ventricular parameters, end systolic diameter (ESD) in #14, #17 and TB4 treated hearts was significantly decreased, when end diastolic dimensions remained unaltered in the peptide treated groups after three weeks, suggesting increased myocyte contractility (FIGS. 2A-D). Masson's trichrome staining at six levels of sections indicated significant reduction of the scar (FIGS. 2E-G and 2M) and increased myocyte diameter in the ischemic area (FIGS. 2I-K and N) after #14, #17, and TB4 treatment when compared to PBS (FIGS. 2H, 2L, 2M and 2N).

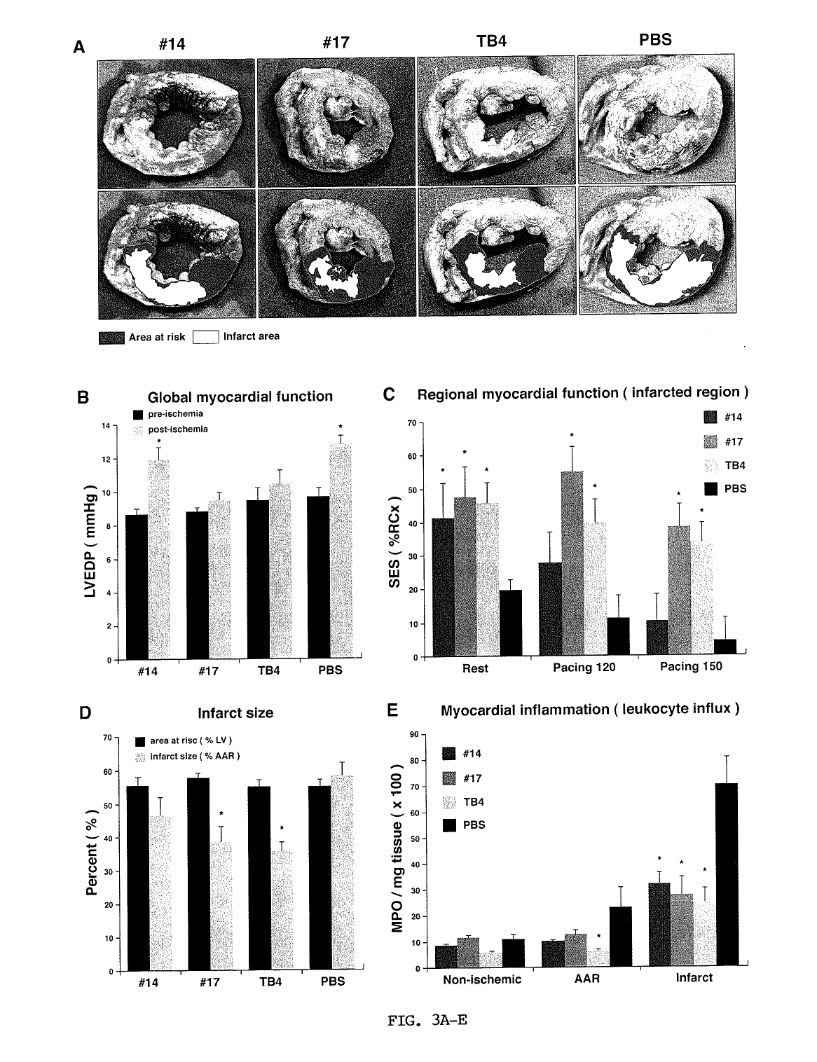

In a preclinical pig model of acute myocardial infarction and reperfusion, regional application of the full-length TB4 at the onset of reperfusion significantly reduced infarct size (35+/−3% (n=5)) compared to control animals (58+/−4% (n=5)). The application of peptide #17 at the same time caused comparable infarct size reduction (38+/−4% (n=5)) as the full-length peptide, whereas infusion of peptide #14 did not reach significance (46±5% (n=5)) (FIGS. 3A, 3D). The area at risk compared to left ventricle did not differ between the four groups (FIG. 3D). These results are reflected in the global (FIG. 3B) and regional (FIG. 3C) myocardial function. The expected increase of left ventricular end-diastolic pressure in the control group (12.8+/−0.5 mmHg) after ischemia and reperfusion was abolished after retroinfusion of full-length TB4 or peptide #17 (10.4+/−0.8 mmHg for TB4 and 9.5+/−0.4 mmHg for peptide #17), but not for peptide #14 (11.9+/−0.8 mmHg). Analysis of the regional myocardial function in the infarcted area revealed similar effects. The diminished function in the control group was significantly improved in the TB4 and peptide #17 groups at rest as well as under increased heart rate, whereas peptide #14 induced increased myocardial function only at rest but not at higher heart rates. Reduced infarct size and improved myocardial function was associated with a reduced post-ischemic inflammatory leukocyte influx into the ischemic heart tissue after TB4 or peptide #17 and #14 application (FIG. 3E). The inventors did not observe any major side effects following #17 and #14 administration in either of our animal models.

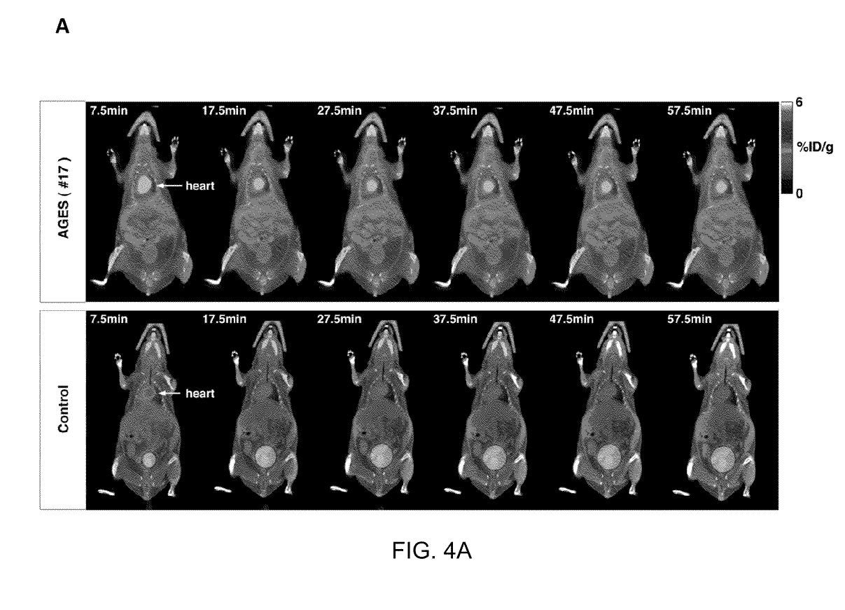

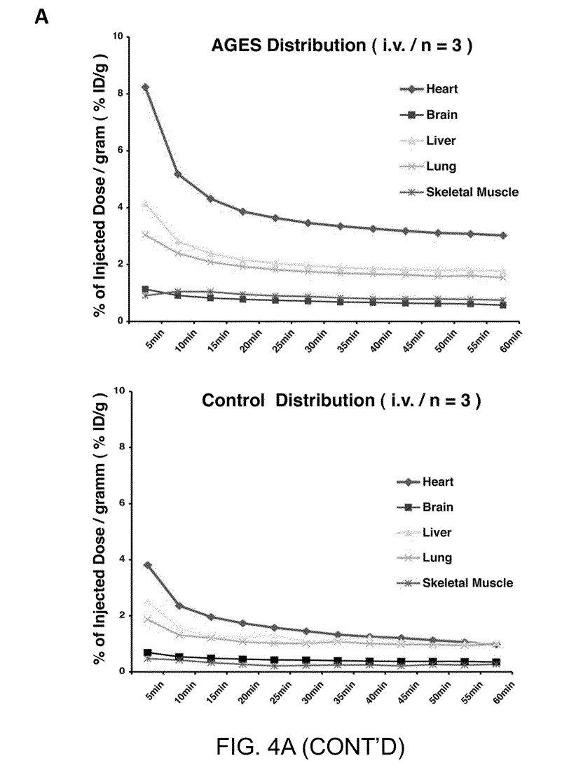

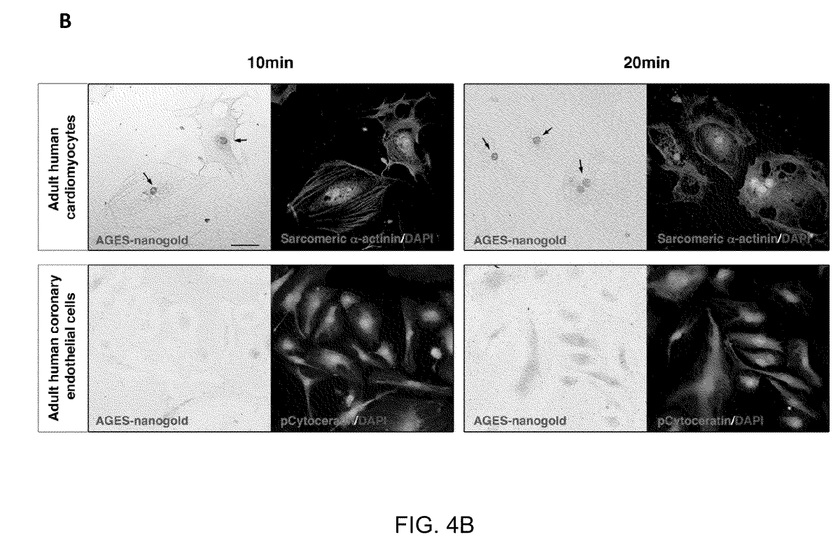

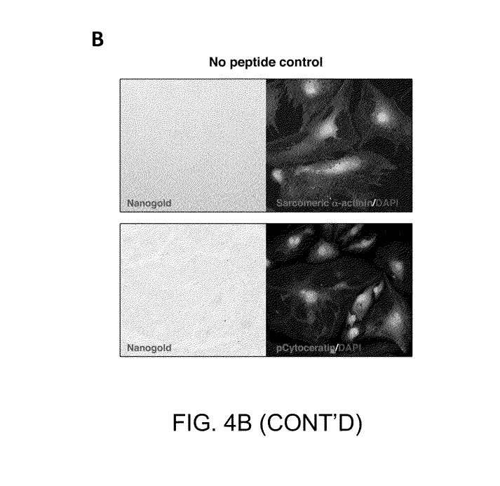

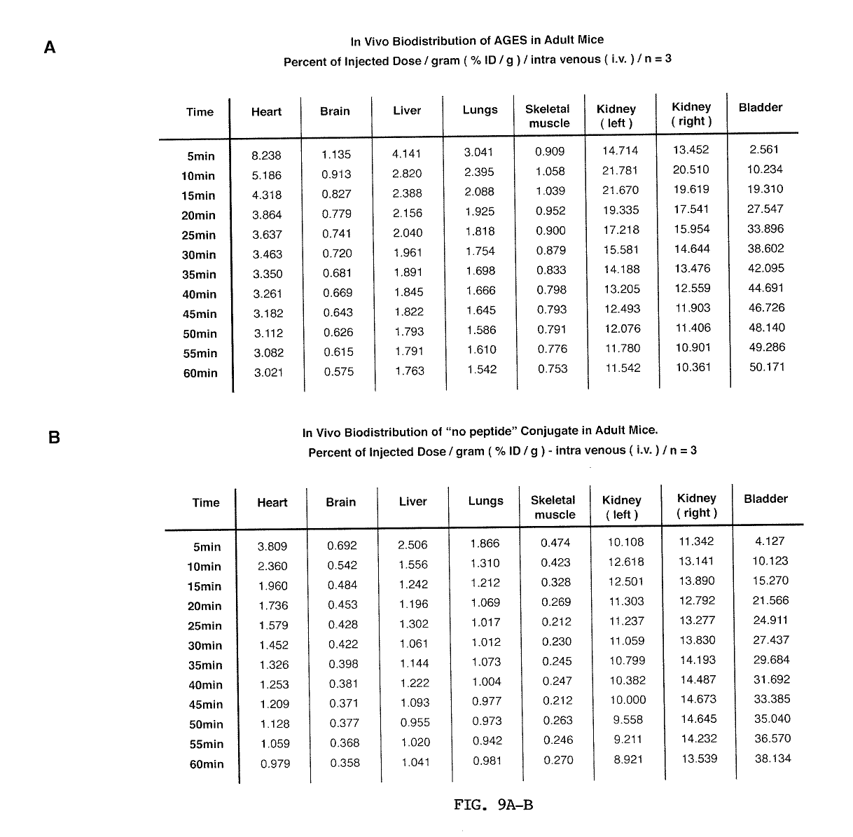

In vivo biodistribution studies revealed specific uptake of AGES into the heart after systemic (i.v.) injection (FIG. 4A; FIG. 9A-B). Supporting our in vivo findings, they detected nanogold labeled AGES in the nuclei of adult human cardiomyocytes (FIG. 4B arrows) ten minutes after external peptide administration in vitro. In contrast to cardiomyocytes, adult human coronary endothelial cells internalized AGES primarily into their cytoplasm after twenty minutes of incubation (FIG. 4B).

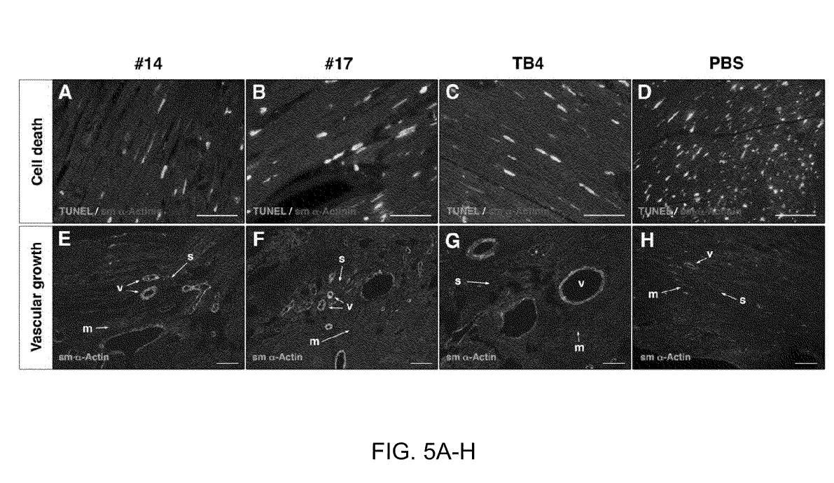

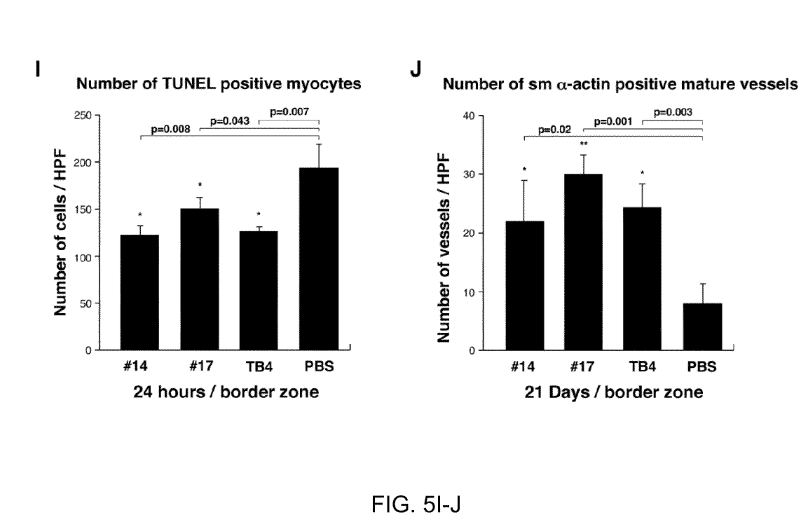

Peptides #14 and #17 affect cell death and post-ischemic vessel growth. Consistent with numerous findings (Simons et al., 2002; Syed et al., 2004; Henry et al., 2000; Losordo et al., 2002), our data suggest drugs may alter heart function distinctively in small and large mammals. To understand this contradiction, the inventors hypothesized, that pathological and molecular comparisons of #14 and #17 may elucidate some of the discriminative elements of heart remodeling between the species. Analyzing the effect on post-ischemic cellular death, they found both peptides significantly decreased TUNEL positivity (FIGS. 5A-D (green) and 5E) in cardiomyocytes (double-labeling with sarcomeric α-actinin antibody (red)) twenty-four hours after ligation. Similar to full-length TB4, this was accompanied by an increase in Akt activation (phosphoS473-Akt) (FIG. 5K), suggesting no differences during early myocyte protection between the constructs.

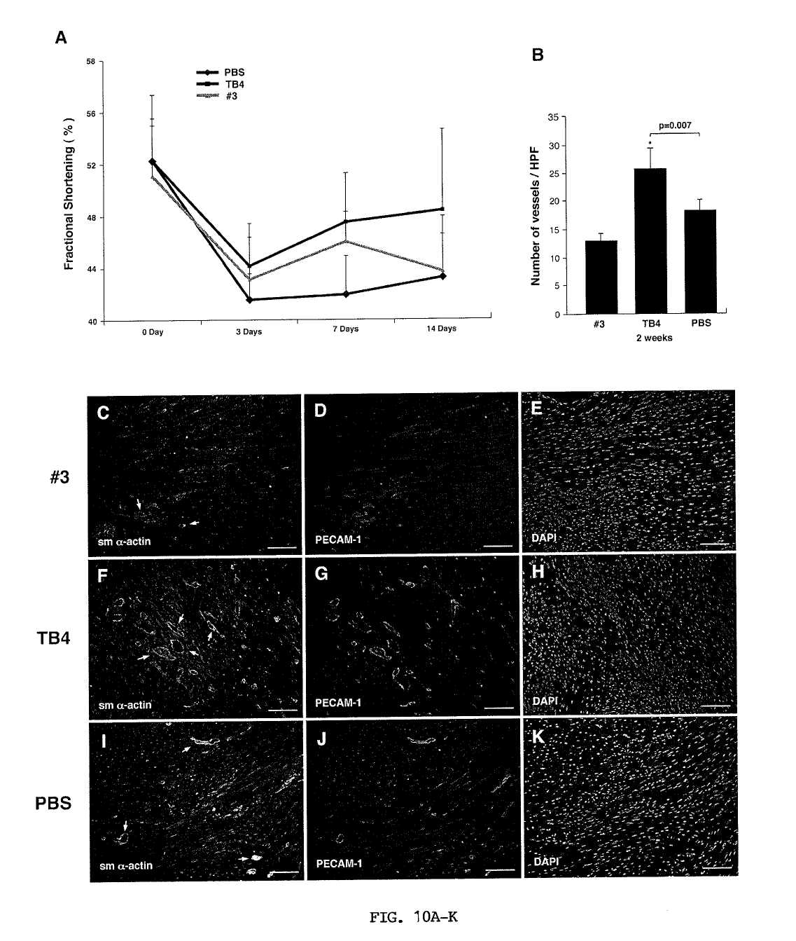

Proper angiogenic response after infarction is believed to be critical for healing and repair. Previous works suggest important role for full-length TB4 in this process (Bock-Marquette et al., 2009; Smart et al., 2007). Comparison of TB4, #14 and #17 to PBS demonstrated all three peptides increase the number of mature, smooth muscle a-actin positive vessels at the infarction border zone after three weeks (FIGS. 5F-I and 5J). VEGF expression and indirect detection of PKC activity by phospho-Marcks (p-Marcks) primary antibodies suggest similar molecular mechanisms for #14, #17 and TB4 during vessel growth (FIG. 5L), most likely by activating the adult epicardium as earlier described (Bock-Marquette et al., 2009). Systemic injection of Ac-SDKPDMox, however, failed to aid cardiac function (FIG. 10A) and did not initiate vessel growth at the border of the infarction (FIGS. 10B-K), supporting our criteria for in vitro candidate selection.

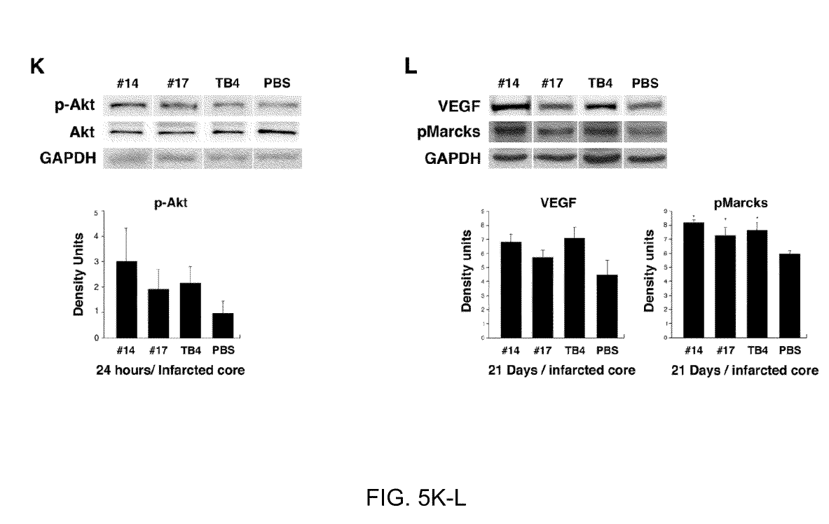

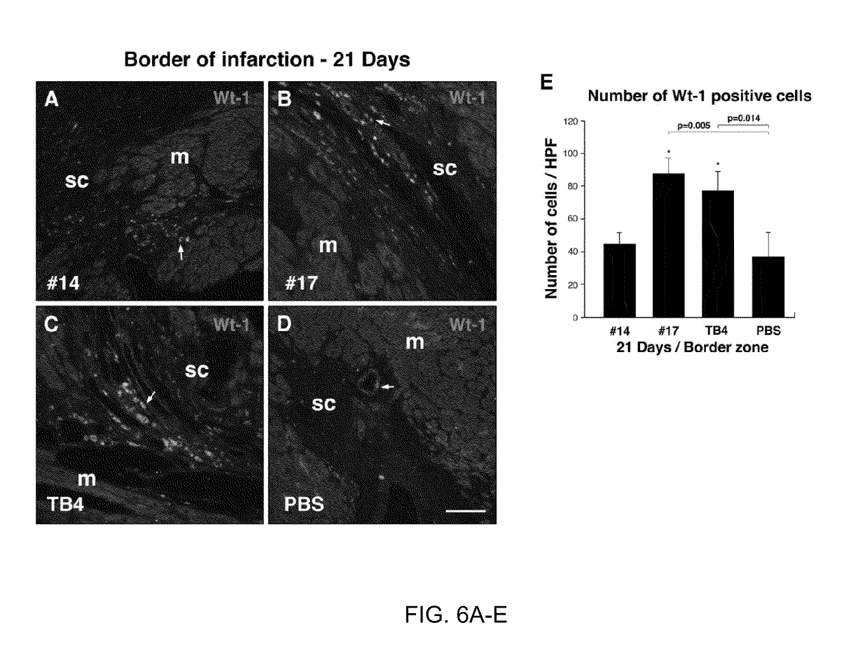

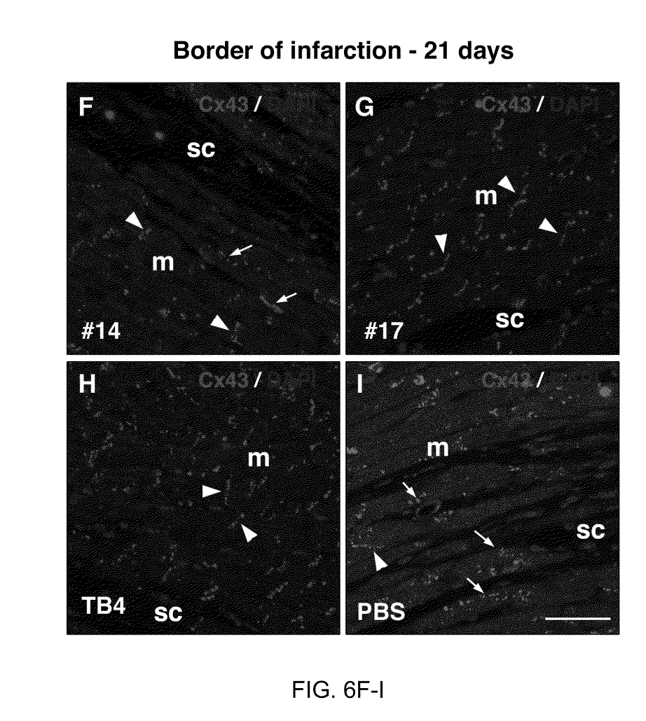

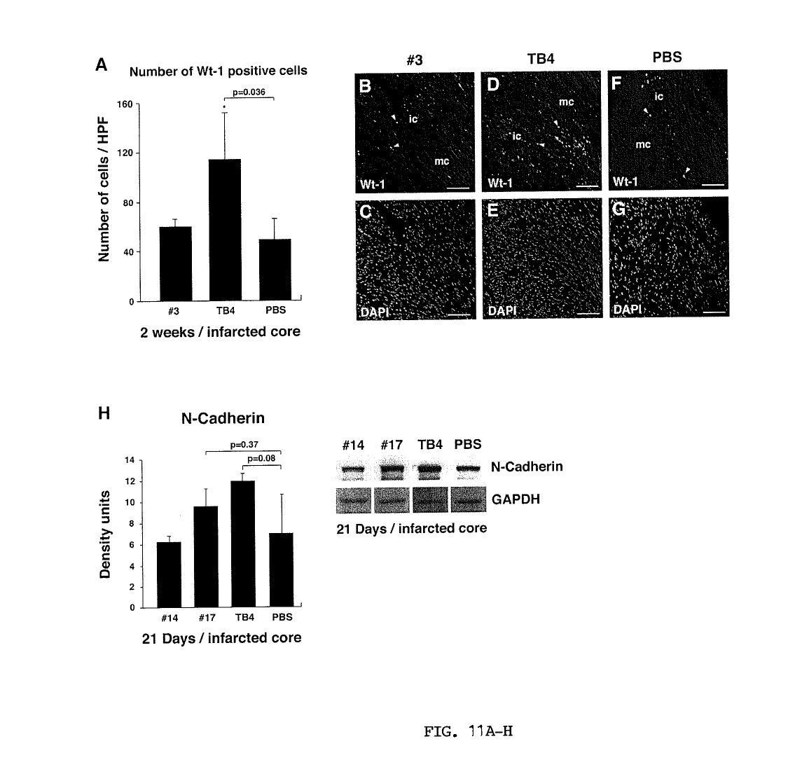

Connexin43 distribution and cardiac progenitor activation may be discriminatory to predict post-ischemic functional recovery in large mammals. The inventors' recent studies suggest TB4 supports long-term post-ischemic muscle repair by myocardial progenitor activation in adult mouse hearts (Bock-Marquette et al., 2009). Thus, the inventors investigated whether AGES is capable of such activation and if #14 and #17 may differ in this respect. They performed immunohistochemical analysis on post-ischemic hearts three weeks after peptide injections. The number of Wt-1 positive cells increased significantly at the border of the infarction when using AGES and TB4 but not after #14 and #3 treatments (FIGS. 6A-E; FIGS. 11A-G). The inventors did not detect alterations in other progenitor markers in the injected hearts (data not shown).

While endogenous myocardial progenitor cell activation may be important to amend post-ischemic repair, it is most likely insufficient to fully account for the observed ameliorations. Coordinated contraction of the heart requires not only higher number of myocytes but cells to be mechanically and electrically coupled. This is maintained by a specialized structure at the ends of myocytes, referred as intercalated discs, containing high concentration of three major intercellular junctional proteins (Jansen et al., 2010). Desmosomes and fascia adherens junctions are essential for mechanical coupling and reinforcing cardiomyocytes, whereas gap junctions are necessary for rapid electrical transmission between cells (Sheikh et al., 2009). In vitro analyses with constructs #14 and #17 suggest AGES increases endothelial cell migration and myocyte beating frequency in larger scale than the actin-binding and helix domain extended peptide #14 (FIGS. 1A-C). Since there were no alterations in cell death, scar volume, vessel number or inflammation between the two constructs, the inventors hypothesized effect on myocyte beating, which relates to improved cell-cell communication and intact current flow causes the functional distinction between #14 and #17 treatments in pigs and mice.

The inventors analyzed N-Cadherin expression, an essential member of the fascia adherens complex, which revealed a slight, but non-significant increase in TB4 and #17 treated animals and showed no changes after #14 injection at the infarction core and border zone by Western blot (FIG. 11H). This suggests a tendency for restoration of intercellular communication in TB4 and #17 treated mice.

To detect alterations in current flow, the inventors investigated the expression and activation of Connexin43 (Cx43), one of the most characterized gap junction proteins of the mammalian heart (Jansen et al., 2010). Ischemic injury decreases Cx43 expression and changes protein localization from intercellular gap junctions to the lateral side of myocytes. These alterations are believed to influence cardiac function (Solan and Lampe, 2009). Western blot analysis on ischemic hearts revealed a significant increase in Cx43 expression after #17 and TB4 injections but not in #14 and PBS treated infarcted cores (FIG. 6J). The inventors did not to detect shifts between #14, #17, TB4 and PBS treated bands most likely because all samples originated from hypoxic tissue. There were no alterations in Cx43 expression in the healthy remote areas (data not shown). Immunohistology using Cx43 specific primary antibody supported Western blot results (FIGS. 6F-I). Outside of expression level, Cx43 also showed notable localization differences after peptide treatments. In PBS and in #14 injected hearts Cx43 localized mainly at the lateral borders of myocytes (FIGS. 6F and 6I white arrows), while after TB4 and #17 injection, Cx43 stayed primarily at the intercalated discs, similar to non-ischemic cells (FIGS. 6G-H white arrowheads). Earlier data suggest Cx43 channel is regulated by kinase (primarily Protein Kinase C) dependent phosphorylation of Cx43 at S368 after ischemia, and the phosphorylation state of Cx43 could be critical and is characteristic for myocyte function (Ek-Vitorin et al., 2006). The inventors found pS368-Cx43 was significantly lower in TB4 and #17 treated hearts (similar to non-ischemic state) but higher after #14 injection in the ischemic border when compared to PBS (FIG. 6J). Our data suggest screening for cardiomyocyte communication (Cx43 and pS368-Cx43 state) in rodent models may be beneficial to predict post-ischemic functional recovery in large mammals. The significance of this observation is supported by the dramatic decrease in regional myocardial function under increased pacing conditions in #14 treated pigs (FIG. 3C blue).

In conclusion, the results above identify a novel tetrapeptide with high therapeutic potential. Systematic investigations of domains and domain combinations of the 43 amino acid secreted peptide TB4 revealed, the C-terminal variable domain AGES significantly increases function in post-ischemic mice and pigs. The physiological and molecular effects of AGES are similar to full-length TB4, suggesting peptide #17 may be regulatory in the initial binding of TB4 to its putative extra- or intracellular receptors or targets.

The inventors extended investigations suggest combined inhibition of initial myocyte death, suppression of inflammation and increased number of coronary vessels per se may be satisfactory in mice but may be insufficient in large mammals. Nonetheless, experiments with constructs #14, #17 and full length TB4 unraveled signs of post-hypoxic cell-cell communication restoration (e.g., Connexin43 activation and expression) could indicate higher success in human studies.

A proper number of functional coronary vessels is critical for post-ischemic recovery (Lavine and Ornitz, 2009). Ac-SDKP (#1) was highly angiogenic but did not aid global function (Yang et al., 2004). The inventors observed a significant increase in the number of mature vessels after #14 treatment. Nonetheless, the construct affected endothelial cell migration to a lesser extent than full-length TB4, #1 or #17. These findings suggest besides late initiation of vascular growth, prevention of early vessel loss could alternatively support cardiac restoration. In addition, initial gene expression analyses on ischemic heart tissues revealed, #14 affects several of the TB4 and #17 activated molecular signaling pathways but in a significantly lower extent (data not shown). These results indicate, that utilization of TB4 domain constructs have the potential to reveal uncharacterized molecular and cellular targets of cardiac regenerative therapy.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.
U.S. Pat. No. 3,817,837
U.S. Pat. No. 3,826,364
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,284,412
U.S. Pat. No. 4,366,241
U.S. Pat. No. 4,472,509
U.S. Pat. No. 4,498,766
U.S. Pat. No. 4,661,913
U.S. Pat. No. 4,680,338
U.S. Pat. No. 4,714,682
U.S. Pat. No. 4,767,206
U.S. Pat. No. 4,774,189
U.S. Pat. No. 4,857,451
U.S. Pat. No. 4,938,948
U.S. Pat. No. 4,989,977
U.S. Pat. No. 5,021,236
U.S. Pat. No. 5,141,648
U.S. Pat. No. 5,160,974
U.S. Pat. No. 5,196,066
U.S. Pat. No. 5,478,722
U.S. Pat. No. 5,563,250
U.S. Pat. No. 5,856,456
U.S. Pat. No. 5,880,270
U.S. Patent Ser. 61/308,618
U.S. Patent Pubin. 2005/0164382
Antibodies: A Laboratory Manual, Cold Spring Harbor Laboratory, 1988.
Atherton et al., Biol. of Reproduction, 32:155-171, 1985.
Ball and Mascagni, Int. J. Pept. Protein Res., 48:31-47, 1996.
Ball and Mascagni, J. Pept. Sci., 3:252-260, 1997.
Berberian et al., Science, 261:1588-1591, 1993.
Bock-Marquette et al., J. Mol. Cell Cardiol., 46:728-738, 2009.
Bock-Marquette et al., Nature, 432:466-472, 2004.
Cleary et al., Trends Microbiol., 4:131-136, 1994.
Dholakia et al., J. Biol. Chem., 264, 20638-20642, 1989.
Eisenwiener et al., Bioconjugate Chemistry 13: 530-541, 2002.
Ek-Vitorin et al., Circ. Res., 98:1498-1505, 2006.
Garner et al., Am. J. Physiol. Heart Circ. Physiol., 285:H2500-2509, 2003.
Grillon et al., FEBS Lett, 274:30-34, 1990.
Henry et al., Int. J. Impot. Res., 12:334-339, 2000.
Hertzog et al., Cell, 117:611-623, 2004.
Hinkel et al., Circulation, 117:2232-2240, 2008.
Huff et al., Eur. J. Biochem., 230:650-657, 1995.
Jansen et al., J. Mol. Cell Cardiol., 48:76-82, 2010.
Kang et al., Science, 240:1034-1036, 1988.
Khatoon et al., Ann. of Neurology, 26, 210-219, 1989.
King et al., J. Biol. Chem., 269, 10210-10218, 1989.
Kohler et al., Methods Enzymol., 178:3, 1989.
Kreier et al., In: Infection, Resistance and Immunity, Harper and Row, New York, 1991.
Kupatt et al., Circulation, 112:1117-122, 2005.
Lavine and Ornitz, Circ. Res., 104:159-169, 2009.
Lenert et al., Science, 248:1639-1643, 1990.
Liu et al., Blood, 101:3014-3020, 2003.
Losordo et al., Circulation, 105:2012-2018, 2002.
Mora et al., Int. J. Immunopharmacol., 19:1-8, 1997.
O'Shannessy et al., J. Immun. Meth., 99, 153-161, 1987.
Owens and Haley, J. Biol. Chem., 259:14843-14848, 1987.
PCT Appln. PCT/US99/17282
PCT Appln. WO 99/49883
Potter and Haley, Meth. in Enzymol., 91, 613-633, 1983.
Rieger et al., Biochem. J., 296(Pt 2):373-378, 1993.
Runyan and Markwald, Dev. Biol., 95:108-114, 1983.
Sasso et al., J. Immunol., 142:2778-2783, 1989.
Sheikh et al., Trends Cardiovasc. Med., 19:182-190, 2009.
Shorki et al., J. Immunol., 146:936-940, 1991.
Silvermann et al., J. Clin. Invest., 96:417-426, 1995.
Simons et al., Circulation, 105:788-793, 2002.
Smart et al., Ann. NY Acad. Sci., 1112:171-188, 2007.
Smart et al., Nature, 445:177-182, 2007.
Solan and Lampe, Biochem. J., 419:261-272, 2009.
Sosne et al., FASEB J., 24(7):2144-2151, 2010.
Syed et al., Cardiology, 101:131-143, 2004.
Yang et al., Hypertension, 43:229-236, 2004.

What is claimed:

1. A peptide of less than 15 residues in length and comprising the sequence AGES, SDKP or both.

2. The peptide of claim 1, wherein said peptide comprises QAGES.

3. The peptide of claim 1, wherein said peptide comprises KQAGES.

4. The peptide of claim 1, wherein said peptide comprises EKQAGES.

5. The peptide of claim 1, wherein said peptide comprises QEKQAGES.

6. The peptide of claim 1, wherein said peptide comprises less than 13, less than 12, less than 11, less than 10, less than 9, less than 8, less than 7, or less than 6 residues.

7. The peptide of claim 1, wherein said peptide consists of LKKTETQEKQAGES.

8. The peptide of claim 1, wherein said peptide consists of AGES, SDKP or SDKPAGES.

9. The peptide of claim 1, wherein said peptide partially or wholly comprises D amino acid residues.

10. The peptide of claim 1, wherein the peptide comprises a SDKPDM motif, and optionally including a modification selected from the group deacetylization, methionine oxidation

11. A method of promoting cardiac repair comprising administering to a subject a peptide of less than 15 residues in length and comprising the sequence AGES, SDKP or both.

12. The method of claim 11, wherein said peptide comprises QAGES.

13. The method of claim 11, wherein said peptide comprises KQAGES.

14. The method of claim 11, wherein said peptide comprises EKQAGES.

15. The method of claim 11, wherein said peptide comprises QEKQAGES.

16. The method of claim 11, wherein said peptide consists of LKKTETQEKQAGES.

17. The method of claim 11, wherein said peptide comprises a SDKPDM motif, and optionally including a modification selected from the group deacetylization, methionine oxidation

18. The method of claim 11, wherein said subject has suffered a myocardial infarct.

19. The method of claim 11, wherein administering comprises intravenous, intraarterial, intracardiac, ocular, or topical administration.

20. The method of claim 11, wherein administering comprises multiple administrations.

21. The method of claim 20, wherein multiple administrations are provided over 3, 4, 5, 6, 7, 14, 21 or 28 days.

22. The method of claim 18, wherein multiple administrations are provided over 3, 4, 5, 6, 7, 14, 21 or 28 days post-infarct.

23. The method of claim 11, wherein administration comprises continuous infusion over a period of 1-24 hours.

24. The method of claim 18, wherein administration comprises continuous infusion over a period of 1-24 hours post-infarct.

25. The method of claim 11, further comprising assessing cardiac function in said subject.

26. The method of claim 11, further comprising providing to said subject a second cardiac therapeutic agent.

27. The method of claim 26, wherein said subject has suffered a myocardial infarct, and said second therapeutic agent is oxygen, aspirin, glyceryl nitrate, a thrombolytic agent, a β blocker, an anticoagulant, or an antiplatelet agent.

28. A method of activating a progenitor cell comprising contacting said cell with a peptide of less than 15 residues in length and comprising the sequence AGES, SKDP or both.

29. The method of claim 28, wherein said progenitor cell is a heart progenitor cell, a brain progenitor cell, a lung progenitor cell, a skeletal muscle progenitor cell, a kidney progenitor cell, a liver progenitor cell, a pancreatic progenitor cell, a spleen progenitor cell, a skin progenitor cell, or a bone marrow progenitor cell.

30. A method of reducing inflammation in a subject comprising administering to said subject a peptide of less than 15 residues in length and comprising the sequence AGES, SKDP or both.

31. A method of reducing the size of a myocardial infarct comprising administering to a subject that has suffered a myocardial infarct a peptide of less than 15 residues in length and comprising the sequence AGES, SKPD or both.
